SEQUENCE LISTING (242 sequences)

SEQ ID NO: 1 - protein, 124 aa, Homo sapiens
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARTPLSLRLRYNWYFDLWGRGTLVTVSS

SEQ ID NO: 2 - protein, 110 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 3 - protein, 119 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVRGSYYLFDYWGQGTLVTVSS

SEQ ID NO: 4 - protein, 109 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPSLTFGGGTKVEIK

SEQ ID NO: 5 - protein, 122 aa, Homo sapiens
QVQLVQSGSELKKPGASVKLSCQASGYSFTNHAMNWVRQAPGQGLEWMGWINTNTGNPTYAQGFTGRFVFSLDTSVSTTYLHISSLKAEDTAVYYCARASARPGVATNLDFWGQGTLVVVSS

SEQ ID NO: 6 - protein, 107 aa, Homo sapiens
DIQMTQSPSSLSASVGDRVTITCRASQNIDNYLNWYQQKPGKAPKLLIYAASRLHSGVPSRFSGSGSGTDFTLIISSLQPEDLATYYCQQSYSNPLTFGPGTKVDIR

SEQ ID NO: 7 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSISGSYLGAFDYWGQGTLVTVSS

SEQ ID NO: 8 - protein, 105 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYTFGQGTKLEIK

SEQ ID NO: 9 - protein, 124 aa, Homo sapiens
QVQLVQSGAEVKKPGASVKVTCKASGYTFTSYYLHWVRQAPGQGLEWMGIINPSGGTTSYAQKFQGRVTMTRDTSTSTVYMELSSLRSEDTAVYYCARAGRRYSSSDDGAFDIWGQGTMVTVSS

SEQ ID NO: 10 - protein, 113 aa, Homo sapiens
DIVMTQSPDSLAVSLGERATINCKSSQNVLYSSNNKNYLAWYQQKPGQPPKLLIYWASTRESGVPDRFSGSGSGTDFTLTISSLQAEDVAVYYCQQYYITPYTFGQGTKLEIK

SEQ ID NO: 11 - protein, 125 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATGPPYCSSTSCEHWFDPWGQGTLVTVSS

SEQ ID NO: 12 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSSYTFGQGTKLEIK

SEQ ID NO: 13 - protein, 121 aa, Homo sapiens; identical to SEQ ID NO: 7

SEQ ID NO: 14 - protein, 105 aa, Homo sapiens; identical to SEQ ID NO: 8

SEQ ID NO: 15 - protein, 132 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARLVPTWASYYDFWSGYPGGYGMDVWGQGTTVTVSS

SEQ ID NO: 16 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPGTFGQGTRLEIK

SEQ ID NO: 17 - protein, 125 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCATHIVVVTATPNWYFDLWGRGTLVTVSS

SEQ ID NO: 18 - protein, 110 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSISTWVFGGGTKLTVL

SEQ ID NO: 19 - protein, 112 aa, Homo sapiens
QVQLQESGPGLVKPSETLSLTCNVSGGSISSYYWSWIRQPPGKGLEWIGYIYYSGSTNYNPSLKSRVTISVDTSKNQFSLKLSSVTAADTAVYYCARGFDYWGQGTLVTVSS

SEQ ID NO: 20 - protein, 108 aa, Homo sapiens
EIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIK

SEQ ID NO: 21 - protein, 118 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARETTGHFDYWGQGTLVTVSS

SEQ ID NO: 22 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPIFTFGPGTKVDIK

SEQ ID NO: 23 - protein, 120 aa, Homo sapiens
QVQLVESGGGVVQPGRSLRLSCAASGFNLSNYAMHWVRQASGKGLEWVSLISYDGSVKYYTDSVKGRFTVSGDNSKNTLFLQMNSLRPDDSALYYCVRGGVSGPNSFDMWGQGTTVTVSS

SEQ ID NO: 24 - protein, 112 aa, Homo sapiens
DVVMTQSPLSLPVTLGQPATISCRSSQSLVYSDGNTYVNWFQQRPGQSPRRLIYQVSIRASGVPDRFSGSGSGTDFALKISRVEAEDVGVYYCMQGTHWPVTFGQGTKVEIK

SEQ ID NO: 25 - protein, 122 aa, Homo sapiens; identical to SEQ ID NO: 5

SEQ ID NO: 26 - protein, 107 aa, Homo sapiens; identical to SEQ ID NO: 6

SEQ ID NO: 27 - protein, 122 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPTVTTITYYFDYWGQGTLVTVSS

SEQ ID NO: 28 - protein, 112 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLNVVFGGGTKLTVL

SEQ ID NO: 29 - protein, 126 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDFNSYQLLWYYYYGMDVWGQGTTVTVSS

SEQ ID NO: 30 - protein, 110 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTWVFGGGTKLTVL

SEQ ID NO: 31 - protein, 125 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQSSFYSSGWYSYGMDVWGQGTTVTVSS

SEQ ID NO: 32 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRGTFGQGTKVEIK

SEQ ID NO: 33 - protein, 124 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARVDYGSGSYGWGWFDPWGQGTLVTVSS

SEQ ID NO: 34 - protein, 110 aa, Homo sapiens
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTYVFGTGTKVTVL

SEQ ID NO: 35 - protein, 122 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATTSPIVGAITWFDPWGQGTLVTVSS

SEQ ID NO: 36 - protein, 111 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGPVFGGGTKLTVL

SEQ ID NO: 37 - protein, 127 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREYYYDSSVYPYYYYAMDVWGQGTTVTVSS

SEQ ID NO: 38 - protein, 110 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPKYTFGQGTKLEIK

SEQ ID NO: 39 - protein, 120 aa, Homo sapiens
EVQLVESGGRLVKPGGSLRLSCAASGITFNNAWMSWVRQAPGKGLEWIARVKSTANGGTIKYAAPVKGRFIISRDVSMNTLSLQMNSLKVEDTAMYYCTTAPHDDYSKNWGQGTLVTVSP

SEQ ID NO: 40 - protein, 108 aa, Homo sapiens
DIQMTQSPSTLSASVGDRVTITCRASQNVGLWLAWYQQKPGKAPKLLIYGASTLQTGVPSRFSGSDSGTECTLTISSLQPDDSATYYCQQYNSDLGITFGQGTRLEIK

SEQ ID NO: 41 - protein, 127 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRVYDYIWGSYRYLDYWGQGTLVTVSS

SEQ ID NO: 42 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 43 - protein, 128 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRESYDILTGYSMEGCFDYWGQGTLVTVSS

SEQ ID NO: 44 - protein, 111 aa, Homo sapiens
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGIRVFGGGTKLTVL

SEQ ID NO: 45 - protein, 126 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARVWGYCSGGSCYVDAFDIWGQGTMVTVSS

SEQ ID NO: 46 - protein, 108 aa, Homo sapiens
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGGGTKLTVL

SEQ ID NO: 47 - protein, 122 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAPPYSPPSSWFDPWGQGTLVTVSS

SEQ ID NO: 48 - protein, 111 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSASYVFGTGTKVTVL

SEQ ID NO: 49 - protein, 130 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCARDHHYDFWSGYSSYYYYGMDVWGQGTTVTVSS

SEQ ID NO: 50 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPYTFGQGTKLEIK

SEQ ID NO: 51 - protein, 134 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDEAYYDILTGYINAPKNYYYYGMDVWGQGTTVTVSS

SEQ ID NO: 52 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPFTFGPGTKVDIK

SEQ ID NO: 53 - protein, 121 aa, Homo sapiens
QVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARSFGGSYYYGMDVWGQGTTVTVSS

SEQ ID NO: 54 - protein, 111 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVTVL

SEQ ID NO: 55 - protein, 118 aa, Homo sapiens
EVQLVESGGGLIQPGGSLRLSCAASGVIVSSNYMSWVRQAPGKGLEWVSVIYSGGSTYYADSVKGRFTISRDNSKNTLYLQMNSLRAEDTAVYYCARDLDVSGGMDVWGQGTTVTVSS

SEQ ID NO: 56 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPQTFGQGTKLEIK

SEQ ID NO: 57 - protein, 122 aa, Homo sapiens; identical to SEQ ID NO: 47

SEQ ID NO: 58 - protein, 111 aa, Homo sapiens; identical to SEQ ID NO: 48

SEQ ID NO: 59 - protein, 122 aa, Homo sapiens
QVQLVQSGAEVKKPGASVKVSCKASGYTFTGYYMHWVRQAPGQGLEWMGWINPNSDVTNYAQKFQGRVTMTRDTSISTAYMELSRLRSDDTAVYYCAREARDYYGSGSLDYWGQGTLVTVSS

SEQ ID NO: 60 - protein, 110 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 61 - protein, 123 aa, Homo sapiens
QVQLVQSGAEVKKPGASVKVSCKASGYTFTSYDISWVRQAPGQGLEWMGWISAYNGNTNYAQKLQGRVTMTTDTSTSTAYMELRSLRSDDTAVYYCARDSVAGIYYYYGMDVWGQGTTVTVSS

SEQ ID NO: 62 - protein, 112 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGPVVFGGGTKLTVL

SEQ ID NO: 63 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSYGGSYYYGMDVWGQGTTVTVSS

SEQ ID NO: 64 - protein, 108 aa, Homo sapiens
SYELTQPHSVSVATAQMARITCGGNNIGSKAVHWYQQKPGQDPVLVIYSDSNRPSGIPERFSGSNPGNTATLTISRIEAGDEADYYCQVWDSSSDHVVFGGGTKLTVL

SEQ ID NO: 65 - protein, 124 aa, Homo sapiens; identical to SEQ ID NO: 1

SEQ ID NO: 66 - protein, 110 aa, Homo sapiens; identical to SEQ ID NO: 2

SEQ ID NO: 67 - protein, 125 aa, Homo sapiens
QVQLVQSGAEVKKPGASVKVSCKASGYTFTGYYMHWVRQAPGQGLEWMGWINPNSGGTNYVQKFQGRVTMTRDTSISTAYMELNRLRSDDTAVYFCARDLTTTAGTDYYYGMDVWGQGTTVTVSS

SEQ ID NO: 68 - protein, 108 aa, Homo sapiens
SYELTQPPSVSVSPGQTARITCSGDALPKQYAYWYQQKPGQAPVLVIYKDTERPSGIPERFSGSSSGTTVTLTISGVQAEDEADYYCQSADSSGTYMIFGTGTKVTVL

SEQ ID NO: 69 - protein, 128 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARARVAYDYIWGSYRYKAFDYWGQGTLVTVSS

SEQ ID NO: 70 - protein, 106 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPRFGPGTKVDIK

SEQ ID NO: 71 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAQTAHYSSSFDYWGQGTLVTVSS

SEQ ID NO: 72 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIK

SEQ ID NO: 73 - protein, 120 aa, Homo sapiens
QVQLVESGGGVVQPGRSMRLSCAASGFNLSSYAMHWVRQASGKGLEWVSLISYDGSIKYYADSVKGRFTVSGDNSKNTLFLQMSSLRADDSALYYCVRGGVSGPNAFDIWGQGTTVTVSS

SEQ ID NO: 74 - protein, 112 aa, Homo sapiens
DVVMTQSPLSLPVTLGQPASISCRSSQSLVYSDGNTYVNWFQQRPGQSPRRLIYKVSNRDSGVPDRFSGSGSGTDFALKISRVEAEDVGVYYCMQGTHWPVTFGQGTKVEIK

SEQ ID NO: 75 - protein, 123 aa, Homo sapiens
QVQLVQSGAEVKKPGASVKVSCKASGYTFSSYGISWVRQDPGQGLEWMGWISAYNGNTNYAQKLQGRVTMTTDTSTSTAYMELRSLRSDDTAVYYCARVGLWWLGHPDAFDIWGQGTMVTVSS

SEQ ID NO: 76 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIK

SEQ ID NO: 77 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARTKGGSYFAPFDYWGQGTLVTVSS

SEQ ID NO: 78 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 79 - protein, 122 aa, Homo sapiens
QVQLVQSGAEVKKPGASVKVSCKASGYTFTSYGISWVRQAPGQGLEWMGWISAYNGNTNYAQKVQGRVTMTTDTSTSTAYMELRSLRSDDTAVYYCARDRGYAATFGVFDYWGQGTLVTVSS

SEQ ID NO: 80 - protein, 107 aa, Homo sapiens
DIQMTQSPSSLSASVGDRVTITCRASQSISSYLNWYQQKPGKAPKFLIYAASSLQSGVPSRFSGSGSGTDFTLTISSLQPEDFATYYCQQTYITAFTFGPGTKVDIK

SEQ ID NO: 81 - protein, 117 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVTVVHFDYWGQGTLVTVSS

SEQ ID NO: 82 - protein, 110 aa, Homo sapiens
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNWVFGGGTKLTVL

SEQ ID NO: 83 - protein, 127 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDLTSTSSSPYSYYYGMDVWGQGTTVTVSS

SEQ ID NO: 84 - protein, 108 aa, Homo sapiens
SYELTQPPSVSVSPGQTARITCSGDALPKQYAYWYQQKPGQAPVVVIYKDSERPSGIPERFSGSSSGTTVTLTISGVQAEDEADYYCQSADSSGTYVVFGGGTKLTVL

SEQ ID NO: 85 - protein, 129 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVTVEAIFGVVILPLKNWFDPWGQGTLVTVSS

SEQ ID NO: 86 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLTLYVFGTGTKVTVL

SEQ ID NO: 87 - protein, 120 aa, Homo sapiens
QVQLVESGGGVVQPGRSLRLSCAASGFNLSNYAMHWVRQASGKGLEWVSLISYDGSIKYYTDSVKGRFTVSGDNSKNTLFLQMNSLRPDDSALYYCVRGGVSGPNSFDMWGQGTTVTVSS

SEQ ID NO: 88 - protein, 112 aa, Homo sapiens
DVVMTQSPLSLPVTLGQPASISCRSSQSLVYSDGNTYVNWFQQRPGQSPRRLIYQVSIRASGVPDRFSGSGSGTDFALKISRVEAEDVGVYYCMQGTHWPVTFGQGTKVEIK

SEQ ID NO: 89 - protein, 123 aa, Homo sapiens
QVQLVQSGAEVKKPGASVKVSCKASGYTFTSYGISWVRQAPGQGLEWMGWISAYSGNTNYAQKLQGRVTMTTDTSTSTAYMEVRSLRSDDTAVYYCARVGLWWLGHPDVFDIWGQGTMVTVSS

SEQ ID NO: 90 - protein, 107 aa, Homo sapiens; identical to SEQ ID NO: 76

SEQ ID NO: 91 - protein, 125 aa, Homo sapiens
QVQLVQSGAEVKKPGSSVKVSCKASGGTFSSYTINWVRQAPGQGLEWMGRIIPILGIADYAQKFQGRVTITADKSTSTAYMELSSLRSEDTAVYYCARDLVEDTAMVTGAAAGTWGQGTLVTVSS

SEQ ID NO: 92 - protein, 112 aa, Homo sapiens
DVVMTQSPLSLPVTLGQPASISCRSSQSLVYSDGNTYLNWFQQRPGQSPRRLIYKVSNRDSGVPDKFSGSGSGTDFTLKISRVEAEDVGVYYCMQGTHWPPTFGQGTKVEIK

SEQ ID NO: 93 - protein, 130 aa, Homo sapiens
RLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARYTMSRITIFGVVSKRGDFDYWGQGTLVTVSS

SEQ ID NO: 94 - protein, 108 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPVTFGPGTKVDIK

SEQ ID NO: 95 - protein, 124 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDIMFGDDWLQKQPDYWGQGTLVTVSS

SEQ ID NO: 96 - protein, 108 aa, Homo sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNLYVFGTGTKVTVL

SEQ ID NO: 97 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAIYYCARPYSGSYYAYFDYWGQGTLVTVSS

SEQ ID NO: 98 - protein, 108 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWLALTFGGGTKVEIK

SEQ ID NO: 99 - protein, 125 aa, Homo sapiens
EVQLLESGGGLAQPGGSLRLSCAASGFTFSSYAMSWVRQAPGKGLEWVSAISGSGGNTYYADSLKGRFTISRDNSKSTLYLQMNSLRAEDTAVYYCAKGPMWQWLVRLSLVGDYWGQGTLVTVSS

SEQ ID NO: 100 - protein, 110 aa, Homo sapiens
SYVLTQPPSVSVAPGQTARITCGGNNIGSKGVHWYQQKPGQAPVLVVFDDSDRPSGIPERFSGSNSGNTATLTISRVEAGDEADYYCQVWDSTSDHPGYVFGTGTKVTVL

SEQ ID NO: 101 - protein, 123 aa, Homo sapiens
QLQLQESGPGLVKPSETLSLTCTVSGGSISSSTYYWGWIRQPPGKGLEWIGNIYYTGSTYYNPSLKSRVTISVDTSKNHFSLRLSSVTAADTAVYYCARHNRFWSEDTPFDPWGQGTLVTVSS

SEQ ID NO: 102 - protein, 107 aa, Homo sapiens; identical to SEQ ID NO: 76

SEQ ID NO: 103 - protein, 122 aa, Homo sapiens
QVQLVQSGAEVKKPGSSVKVSCKASGGTFSSYAITWVRQAPGQGLEWMGGIIPIFGTANYAQKFQGRVTITADESTSTAYMELSSLRSEDTAVYYCVRVALYDSSGYSPDYWGQGTLVTVSS

SEQ ID NO: 104 - protein, 109 aa, Homo sapiens
EIVLTQSPATLSLSPGERATLSCRASQSVSNFLAWYQQKPGQAPRLLIYDASNRATGIPARFSGSGSGTDFTLTISSLEPEDFAVYYCQHRSNWPPGITFGQGTRLEIK

SEQ ID NO: 105 - protein, 122 aa, Homo sapiens
QVQLVQSGAEVKKPGSSVKVSCKASGGTFSSYAISWVRQAPGQGLEWMGGIIPIFGTTNNAQKFQGRVTLTADESTSAAYMELSSLRSEDTAVYYCARIEPYASTGYTFDYWGQGTLVTVSS

SEQ ID NO: 106 - protein, 109 aa, Homo sapiens
EIVLTQSPATLSLSPGERATLSCRASQSVSSFLAWYQQKPGQAPRLLIYDASNRATGIPARFSGSGSGTDFTLTISSLEPEDFAVYYCQQRSNWPPRITFGQGTRLEIK

SEQ ID NO: 107 - protein, 121 aa, Homo sapiens
QVQLVESGGGVVQPGRSLRLSCAASGFTFSIYAIHWVRQAPGKGLEWVAVISYDGINKYYADAVKGRFTISRDNSKNTLFLQMNSLRTEDTAVYYCARAMGGNYFHAFDIWGQGTMVTVSS

SEQ ID NO: 108 - protein, 108 aa, Homo sapiens
EIVLTQSPGTLSLSPGERATLSCRASQSVSSSYLAWYQRKPGQAPRLLIYGASNRATGIPDRFSGSGSGTDFTLTISRLEPEDFAVFYCQQYGSSPWTFGQGTKVEIK

SEQ ID NO: 109 - protein, 125 aa, Homo sapiens
EVQLVQSGAEVKKPGESLKISCKGSGNSFTSYWIGWVRQMPGKGLEWMGIIYPGDSDTRYSPSFQGQVTISADKSISTAYLQWSSLKASDTAMYYCARHRNYDILTGYRYYFDYWGQGTLVTVSS

SEQ ID NO: 110 - protein, 108 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKLEIK

SEQ ID NO: 111 - protein, 121 aa, Homo sapiens
QVQLVESGGGVVQPGRSLRLSCGASGFIFSSYAMHWVRQAPGKGLEWVAVISYDGNNKYYADSVKGRFTISRDNSKNTLYLQMNSLRAEDTAVYYCARPYSGTYFSAFDIWGQGTMVTVSS

SEQ ID NO: 112 - protein, 112 aa, Homo sapiens
DIVMTQSPDSLAVSLGERATINCKSSQSVLYSSNNKNYLAWYQLQPGQPPKLLIYWASTRESGVPDRFSGSGSGTDFTLTISSLQAEDVAVYYCQQYYSIPTFGQGTKVEIK

SEQ ID NO: 113 - protein, 115 aa, Homo sapiens
EVQLVESGGGLIQPGGSLRLSCAASGFTVSSNYMTWVRQAPGKGLEWVSIIYSGGSTYYADSVKGRFTISRDNSKNTLYLQMNSLRAEDTAVYYCAAGYYDSINWGQGTLVTVPS

SEQ ID NO: 114 - protein, 108 aa, Homo sapiens
DIQMTQSPSTLSASVGDRVTITCRASQSISTWLAWYQQKPGKAPKLLIYKASNLESGVPSRFSGSGSGTEFTLTISSLQPDDFATYYCQQYYTYSWTFGQGTKLEIKR

SEQ ID NO: 115 - protein, 124 aa, Homo sapiens
QVQLQESGPGLVKPSGTLSLTCAVSGGSISSSTWWSWVRQPPGKGLEWIGEIYHSGSTNYNPSLKSRVTISVDKSKNQFSLKLSSVTAADTAVYYCARSASNYFGYYYYGMDVWGQGTTVTVSS

SEQ ID NO: 116 - protein, 114 aa, Homo sapiens
  misc_feature (110)..(112): Xaa can be any naturally occurring amino acid
  misc_feature (114)..(114): Xaa can be any naturally occurring amino acid
EIVLTQSPATLSLSPGERATLSCRASQSVSSQLAWYQQKPGQAPRLLISDASNRATGIPARFSGSGSGTDFTLTISSLEPEDFAVYYCQQRSNWPRTFGQGTKVEIKRFXXXGX

SEQ ID NO: 117 - protein, 125 aa, Homo sapiens
EVQLVESGGGLVKPGGSLRLSCAASGFTFSSYSMNWVRQAPGKGLEWVSSISSSRSYIYYADSVKGRFTISRDNAKNSLYLQMNSLRAEDTAVYYCASNPAADPNTYYYYGMDVWGQGTTVTVSS

SEQ ID NO: 118 - protein, 116 aa, Homo sapiens
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVGVFGGGTKLTVL

SEQ ID NO: 119 - protein, 136 aa, Homo sapiens
QVQLVESGGGVVQPGRSLRLSCAASGFTFSSYGIHWVRQAPGKGLEWVAVISYDGINKYYADSVKGRFTISRDNSKNTLYLHMNSLRAEDTAVYYCARPNSGSYHSYFDYWGQGTLVTVSSASTKGPSVFPLAPSS

SEQ ID NO: 120 - protein, 108 aa, Homo sapiens
EIVLTQSPATLSLSPGERATLSCGASQSVSSSYLAWYQQKPGLAPRLVIYDASSRATGIPDRFSGSGSGTDFTLTISRLEPEDFAVYYCQQYGSSPQTFGQGTRLEIK

SEQ ID NO: 121 - protein, 124 aa, Homo sapiens
EVQLVESGGGLVQPGGSLRLSCAASGFTFSSYEMNWVRQAPGKGLEWVSYISSSGSTIYYADSVKGRFTISRDNAKNSLYLEMNSLRAEDTAVYYCASTSDSGSYYIHWYFDLWGRGTLVTVSS

SEQ ID NO: 122 - protein, 111 aa, Homo sapiens
QSALTQPASVSGSPGQSITISCTGTSSDVGSYNLVSWYQQHPGKAPKLMIYDVSKRPSGVSNRFSGSKSGNTASLTISGLQAEDEADYYCCSHAGSSTSYIFGTGTKVTVL

SEQ ID NO: 123 - protein, 121 aa, Homo sapiens
QVQLVESGGGVVQPGRSLRLSCAASGFTFSSFALHWVRQAPGKGLEWVAVVSYDGSNKYYADSVKGRFTISRDNSKNTLYLQMNSLRAEDTAAYYCARSMGGNYADYFDYWGQGTLVTVSS

SEQ ID NO: 124 - protein, 107 aa, Homo sapiens
EIVLTQSPATLSLSPGERATLSCRASQSVTNYLAWYQQKPGQAPRLLIYDASNRATGIPARFSGSGSGTDFTLTISSLEPEDFAVYYCQQRSNWPNTFGQGTRLEIK

SEQ ID NO: 125 - protein, 122 aa, Homo sapiens
QVQLVQSGAEVKKPGSSVKVSCKTSGGIFSSYAISWVRQAPGQGLEWMGGIIPIFGTANYAQKFQGRVTITADESTSTAYMELSSLRSEDTAVYYCASYSGFDDYGHYRNYWGQGTLVTVSS

SEQ ID NO: 126 - protein, 109 aa, Homo sapiens
DIVMTQTPATLSLSPGERATLSCRASQSVSRYLAWYQQKPGQAPRLLIYDASNRATGIPARFSGSGSGTDFTLTISSLEPEDFAVYYCQQRYNWPPELTFGGGTKVEIK

SEQ ID NO: 127 - protein, 122 aa, Homo sapiens
QVQLVQSGAEVKKPGSSVKVSCKASGGTFSSFAFIWVRQAPGQGLEWMGGIIPIFGTAKYTQKFQGRVTITADESTSTAYMELSSLRSEDTAVYYCARVSYYDSSGFYTDYWGQGTLVTVSS

SEQ ID NO: 128 - protein, 109 aa, Homo sapiens
DIVMTQSPATLSLSPGERATLSCRASQSVSSFLAWYQQKPGQAPRLLIYDASNRATGIPARFSGSGSGTDFTLTISSLEPEDFAVYYCQQRSNWPPEYTFGQGTKLEIK

SEQ ID NO: 129 - protein, 122 aa, Homo sapiens
EVQLVESGGGLVQPGGSLRLSCSASGFTFSSYAMHWVRQAPGKGLEYVSTISTNGGSTYYADSVKGRFTISRDNSKNTLYLQMSSLRAEDTAVYYCVLGYCSSTSCQHFDYWGQGTLVTVSS

SEQ ID NO: 130 - protein, 111 aa, Homo sapiens
QSALTQPASVSGSPGQSITISCTGTSSDVGAYNYVSWYQQHPGKAPKLMIYEVSNRPSGVSNRFSGSKSGNTASLTISGLQAEDEADYYCSSYTSSSTPYVFGTGTKVTVL

SEQ ID NO: 131 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVGAYDSSGYYQDLWGRGTLVTVSS

SEQ ID NO: 132 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVITFGQGTRLEIK

SEQ ID NO: 133 - protein, 121 aa, Homo sapiens
QVQLVESGGGVVQPGRSLRLSCAASGFTFSSYAMHWVRQAPGKGLEWVAIISYDGINKYYADSVKGRFTISRDNSKNTLYLQMNSLRVEDTAVYYCARPGGGSYYGPFDYWGQGTLVTVSS

SEQ ID NO: 134 - protein, 111 aa, Homo sapiens
QSALTQPASVSGSPGQSITISCTGTSSDVGSYNYVSWYQQHPGKAPKLMIYEVSNRPSGVSNRFSGSKSGNTASLTLSGLQAEDEADYYCSSYTSSSTLYVFGTGTKVTVL

SEQ ID NO: 135 - protein, 126 aa, Homo sapiens
QVQLVQSGAEVKKPGASVKVSCKASGYTFTGYYMHWVRQAPGQGLEWMGWINPNSGGTNYAQKFQGRVTMARDTSISTAYMELSRLRSDDTAVYYCARDQVSPYCGGDCPDAFDIWGQGTMVTVSS

SEQ ID NO: 136 - protein, 110 aa, Homo sapiens; identical to SEQ ID NO: 100

SEQ ID NO: 137 - protein, 128 aa, Homo sapiens
QVQLVQSGAEVKKPGASVKVSCKAFGYTFTKNGINWVRQAPGQGLEWMGWISPNNGYTNFAQKFQGRATMTSDTSTSTAYMELRSLRSDDTAVYYCARVRVDYDSSGYDAYYYHLDVWGQGTTVTVSS

SEQ ID NO: 138 - protein, 110 aa, Homo sapiens
QSVLTQPPSASGTPGQRVTISCSGSSSNIGRNTVNWFQQLPGTAPKLLIYSNYQRPSGVPDRLSGSKSGTSASLAIRGLQSEDEADYYCAAWDDSLHGWVFGGGTKLTVL

SEQ ID NO: 139 - protein, 121 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTALYYCASEIVSFIGSSEDYWGQGTLVTVSS

SEQ ID NO: 140 - protein, 109 aa, Homo sapiens
SYELTQPPSVSVSPGQTARITCSGDALPKKYAYWYQQKSGQAPVLVIYEDSKRPSGIPERFSGSSSGTMATLTISGAQVEDEADYCCYSTDSSDNHRRVFGTGTKVTVL

SEQ ID NO: 141 - protein, 120 aa, Homo sapiens
QVQLVESGGGVVQPGRSLRLSCAASGFTFNSFAMHWVRQAPGKGLEWVAVISYDGNNKYYADSVKGRFTISRDNSKNTLYLQMNSLRAEDTAVYYCARDRSGGYSCFDYWGQGTLVTVSS

SEQ ID NO: 142 - protein, 106 aa, Homo sapiens
DIVMTQSPATLSVSPGERATLSCRASQSVSSKLAWYQQKPGQAPRLLIYGASTRATGIPARFSGSGSGTEFTLTISSLQSEDFAVYYCQQYNNWWTFGQGTKVEIK

SEQ ID NO: 143 - protein, 127 aa, Homo sapiens
EVQLVESGGGLVKPGGSLRLSCAASGFTFSSYSMNWVRQAPGKGLEWVSSISSTSNYIYYADSVKGRFTISRDNAKNSLYLQMNSLRAEDTAVYYCARVSPNYDFWSGYYPDAFDIWGQGTMVTVSS

SEQ ID NO: 144 - protein, 108 aa, Homo sapiens
DIVMTQSPATLSVSPGERATLSCRASQSVSSNLAWYQQKPGQAPRLLIFGASTRATGIPARFSGSGSGTEFTLTISSLQSEDFAVYYCHQYYNWPPWTFGQGTKVEIK

SEQ ID NO: 145 - protein, 121 aa, Homo sapiens
EVQLVQSGAEVKKPGESLRISCKGSGYSFTSYWISWVRQMPGKGLEWMGKIDPSDSYTNYSPSFQGHVTISADKSISTAYLQWSSLKASDTAMYYCARQFGGELLGWFDPWGQGTLVTVSS

SEQ ID NO: 146 - protein, 107 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPFTFGPGTKVDIK

SEQ ID NO: 147 - protein, 118 aa, Homo sapiens
QVQLVESGGGVVQPGRSLRLSCAAAGFIFSSYAMNWVRQAPGKGLEWVAVISYDGSNKYYADSVKGRFTISRDNSKNTLNLQMNSLRAEDTAVYYCARGNGYIIIDYWGQGTLVTVSS

SEQ ID NO: 148 - protein, 110 aa, Homo sapiens
QLVLTQSPSASASLGASVKLTCTLSSGHSSYAIAWHQQQPEKGPRYLMKFNSDGTHSKGDGIPDRFSGSSSGAERSLTISSLQSEDEADYYCQTWGTGGVFGGGTKLTVL

SEQ ID NO: 149 - protein, 126 aa, Homo sapiens
QLQLQESGPGLVKPSETLSLTCTVSGGSISSSSYYWGWIRQPPGKGLEFIGSIYYSGSTYYNPSLKSRVTISVDTSKNQFSLKLSSVTAADTAVYYCATQEVVLVRPYNYNGMDVWGQGTTVTVSS

SEQ ID NO: 150 - protein, 110 aa, Homo sapiens
QSALTQPASVSGSPGQSITLSCTGTSSDVGGYNYVSWYQQHPGKAPKLMIYEVSNRPSGVSNRFSGSKSGNTASLTISGLQAEDEADYYCSSYTSISTLVFGTGTKVTVL

SEQ ID NO: 151 - protein, 123 aa, Homo sapiens
QLQLQESGPGLVKPSETLSLTCTVSGGSVSSNAYYWGWIRQPPGKGLEWIGSVNYRGSTYYIPTLKSRITISVNTSKNQFSLKLTSVTAADTAVYYCARRGVALVTITYFDFWGQGALVTVSS

SEQ ID NO: 152 - protein, 108 aa, Homo sapiens
QSVLTQPPSVSGAPGQRVTITCTGSSSNIGSGYDVHWYQQLPGTAPKLLIYGNTNRPSGVPDRFSGSKSGTSASLAITGLQAEDEAHYYCQSYDSSVVFGGGTKLTVL

SEQ ID NO: 153 - protein, 121 aa, Homo sapiens
QVQLVESGGGVVQPGRSLRLSCAASGFTFSTYAMHWVRQAPGKGLEWVAVISYDGSNKYYADSVKGRFTISRDNSKNTLYLQMNSLRAEDTAVYYCARAYGGSYFYGMDVWGQGTTVTVSS

SEQ ID NO: 154 - protein, 104 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSFGGGTKVEIK

SEQ ID NO: 155 - protein, 122 aa, Homo sapiens
QVQLVQSGAEVKKPGSSVKVSCKASGGTFSSYAITWVRQAPGQGLEWMGGIIPIFGTANYAQKFQGRVTITADESTNTAYMELSSLTSEDTAVYYCAKIGDYDSSGYYFDYWAQGTLVTVSS

SEQ ID NO: 156 - protein, 109 aa, Homo sapiens
EIVLTQSPATLSLSPGERATLSCRASQSVSSFLAWYQQKPGQAPRLLIYDASNRATGIPARFSGSGSGTDFTLTISSLEPEDFAVYYCQQRSNWPPSITFGQGTRLEIK

SEQ ID NO: 157 - protein, 122 aa, Homo sapiens
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTMVWQQLHDAFDIWGQGTMVTVSS

SEQ ID NO: 158 - protein, 109 aa, Homo sapiens
SYELTQPPSVSVSPGQTARITCSGDALPKQYAYWYQQKPGQAPVLVIYKDSERPSGIPERFSGSSSGTTVTLTISGVQAEDEADYYCQSADSSGTYSWVFGGGTKLTVL

SEQ ID NO: 159 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARISSYDSSGYSMDVWGQGTTVTVSS

SEQ ID NO: 160 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRVTFGPGTKVDIK

SEQ ID NO: 161 - protein, 121 aa, Homo sapiens
QVQLVESGGGVVQPGRSLRLSCAASRFTFSTYAMHWVRQAPGKGLEWVTVISYDGDNKYYADSVKGRFTISRDNSKNTLYLQMNSLRTEDTAVYYCTRAGSGSYLSYFDYWGQGTLVTVSS

SEQ ID NO: 162 - protein, 110 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCHHYGSSPPLFTFGPGTKVDIK

SEQ ID NO: 163 - protein, 119 aa, Homo sapiens
EVQLVESGGGLVQPGGSLRLSCAASGFTFSNCWMSWVRQAPGKGLEWVANIKQDGSEKYYVDSVKGRFTISRDNAKNSLYLQMNSLRAEDTAVYYCARMGNTAWYFDLWGRGTLVTVSS

SEQ ID NO: 164 - protein, 106 aa, Homo sapiens
SYELTQPPSVSVSPGQTASITCSGDKLGDKYACWYQQKPGQSPVLVIYQDNKRPSGIPERFSGSNSGNTATLTISGTQATDEADYYCQAWDSSTGVFGTGTKVTVL

SEQ ID NO: 165 - protein, 115 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCATNSNYPYWGQGTLVTVSS

SEQ ID NO: 166 - protein, 108 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYRPWTFGQGTKVEIK

SEQ ID NO: 167 - protein, 119 aa, Homo sapiens
QVQLVQSGAEVKKPGASVKVSCRASGYTFTNFGISWVRQAPGLGLEWMGWISTYNGNTNYAQKLQGRVTMTTDTSTSTAYMELRSLRSDDTAVYYCARASGPYLLGLYWGQGTLVTVSS

SEQ ID NO: 168 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSDVVFGGGTKLTVL

SEQ ID NO: 169 - protein, 129 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASARFITMVRGVINIYYYGMDVWGQGTTVTVSS

SEQ ID NO: 170 - protein, 110 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLLFGGGTKLRSC

SEQ ID NO: 171 - protein, 116 aa, Homo sapiens
EVQLVESGGGLVQPGGSLRLSCSASGFTFSSYAMHWVRQAPGKGLEYVSGISSNGGSTYYADSVKGRITISRDNSKKTLYLQMSSLRAEDTAVYYCVKSSSRGYYWGQGTLVTVSS

SEQ ID NO: 172 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 173 - protein, 120 aa, Homo sapiens
QVQLVESGGGLVKPGGSLRLSCAASGFTFSDYYMSWIRQAPGKGLEWVSYISSRSSYTNYADSVKGRFTISRDNAKNSLYLQMNSLRAEDTAVYYCARGYDSSGYHYSLWGQGTLVTVSS

SEQ ID NO: 174 - protein, 112 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTNSRTLDVVFGGGTKLTVL

SEQ ID NO: 175 - protein, 130 aa, Homo sapiens
QVQLQQWGAGLLKPSETLSLTCAVYGGSFSGYYWSWIRQPPGKGLEWIGEINHSGSTNYNPSLKSRVTISVDTSTNQFSLKLSSVTAADTAVYYCARRVHYDILTGFYGGALITRAFDIWGQGTMVTVSS

SEQ ID NO: 176 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTLHTFGQGTKLEIK

SEQ ID NO: 177 - protein, 127 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDRRGSGWSWDYYYYGMDVWGQGTTVTVSS

SEQ ID NO: 178 - protein, 112 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPRTFGQGTKLEIK

SEQ ID NO: 179 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAYSYDSSGYSPDYWGQGTLVTVSS

SEQ ID NO: 180 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPKLTFGGGTKVEIK

SEQ ID NO: 181 - protein, 127 aa, Homo sapiens
QVQLVQSGAEVKKPGASVKVSCKASGYTFISYGISWVRQAPGQGLEWMGWISGYNGNTNYAQKFQGRVTMTTDTSTSTAYMELRSLRSDDTAVYYCARVRIFGVVIIVDTAMVLDYWGQGTLVTVSS

SEQ ID NO: 182 - protein, 110 aa, Homo sapiens
QSVLTQPPSVSAAPGQKVTISCSGSSSNIGNNYVSWYQQLPGTAPKLLIYENNKRPSGIPDRFSGSKSGTSATLGITGLQTGDEAHYYCGTWDSSLSAGVFGGGTKLTVL

SEQ ID NO: 183 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLDLQMNSLRAEDTAVYYCARAYGGNYYYGMDVWGQGTTVTVSS

SEQ ID NO: 184 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNNYPLTFGGGTKVEIK

SEQ ID NO: 185 - protein, 127 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRLYDYVWGSYRFGHTWGQGTLVTVSS

SEQ ID NO: 186 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPRTFGQGTKVEIK

SEQ ID NO: 187 - protein, 119 aa, Homo sapiens
QVQLVQSGAEVKKPGSSVKVSCKTSGGTFSNYAISWVRQAPGQGLEWMGGIIPIFGTANYAQKFQGRVTFTADESTSTAYMELSSLRSEDTAVFYCVSRIAAAADFDYWGQGTLVTVSS

SEQ ID NO: 188 - protein, 109 aa, Homo sapiens
EIVLTQSPATLSLSPGERATLSCRASQSISSYLAWYQQKPGQAPRLLIYDASNRATGIPARFSGSGSGTDFTLTISSLEPEDFAVYYCQQRSNWPPLLTFGGGTKVEIK

SEQ ID NO: 189 - protein, 126 aa, Homo sapiens
QLQLQESGPGLVKPSETLSLTCTVSGGSISSSSYYWGWIRQPPGKGLEWIGNIYYSGITYYNPSLKSRVTISVDTSKNQFSLKLSSVTAADTAVYYCARQLWGYYDSSGYYAFDIWGQGTMVTVSS

SEQ ID NO: 190 - protein, 111 aa, Homo sapiens
QSALTQPASVSGSPGQSITISCTGTSSDVGSYNLVSWYQQHPGKAPKLMIYEGSKRPSGVSNRFSGSKSGNTASLTISGLQAEDEAAYYCCSYVGSSTYVVFGGGTKLTVL

SEQ ID NO: 191 - protein, 122 aa, Homo sapiens
QVQLQESGPELVKPSETLSLTCSVSGGSISSYYWSWIRQPPGKGLEWIGYIYYSGSTNYNPSLSGSTNYNPSLKSRVTVSVDTSKNQFSLKVSSVTAADTAVYYCARGFDYWGQGTLVTVSS

SEQ ID NO: 192 - protein, 108 aa, Homo sapiens
EIVLTQSPGTLSLSPGERATLSCRASQSVSSIYLAWYQQKPGQAPRLLIYGASSRATGIPDRFSGSGSGTDFTLTISRLEPEDFAVYYCQQYGSSPWTFGQGTKVEIK

SEQ ID NO: 193 - protein, 125 aa, Homo sapiens
QVQLVESGGGVVQPGRSLRLSCAASGFTFSSYGMHWVRQAPGKGLEWVAVISYDGSNQYYADSVKGRFTISRDNSKNTLYLQMNSLRAEDTAVYYCAKDPTIFGVVLQIPRFDYWGQGTLVTVSS

SEQ ID NO: 194 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKLEIK

SEQ ID NO: 195 - protein, 125 aa, Homo sapiens
QVQLVESGGGVVQPGRSLRLSCAASGFTFSSYAMHWVRQAPGKGLEWVAVISYDGINKYYADSVKGRFTISRDSSKNTLYLQMNSLRAEDTAVYYCARGGVGYQLLFGNDWFDPWGQGTLVTVSS

SEQ ID NO: 196 - protein, 111 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDESDYYCSSYTTSSTLYVFGTGTKVTVL

SEQ ID NO: 197 - protein, 122 aa, Homo sapiens
QVQLVQSGAEVKKPGSSVKVSCKASGGTFTSYAITWVRQAPGQGLEWMGGIIPIFGTANYAQKFQGRVTITADESTSTAYMELSSLRSEDTAVYYCARVGAYDSSGYYQDLWGRGTLVTVSS

SEQ ID NO: 198 - protein, 109 aa, Homo sapiens; identical to SEQ ID NO: 132

SEQ ID NO: 199 - protein, 122 aa, Homo sapiens
QVQLVQSGAEVKKPGSSVKVSCKASGGTFSSYTISWVRQAPGQGLEWMGGIIPIFGTANYAQNFQGRVTITADESTSTAYMELSSLRSEDTAVYYCARVSYYDSSGYYTDYWGQGTLVTVSS

SEQ ID NO: 200 - protein, 109 aa, Homo sapiens
EIVLTQSPATLSLSPGERATLSCRASQSVSSFLAWYQQKPGQAPRLLIYDASNRATGIPARFSGSGSGTDFTLTINSLEPEDFAVYYCQQRSNWPPEYTFGQGTKLEIK

SEQ ID NO: 201 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARALTGNYYYGMDVWGQGTTVTVSS

SEQ ID NO: 202 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 203 - protein, 120 aa, Homo sapiens
QVQLVESGGGVVQPGNSLRLSCVASGFKFSSSAMHWVRQAPGKGLEWVAIIWFHGNTKFYGDFVKGRFTVSRDNSNNTLHLQMNSLRAEDTAVYHCARASSDYGGAFDIWGQGTMVTVSS

SEQ ID NO: 204 - protein, 108 aa, Homo sapiens
EIVMTQSPATLSVSLGERATLSCRASQSVTSSLAWYQHKPGQAPRLLIYGASTRATGIPARFSGSGSGTEFTLTISSLQSEDFAVYFCQQYNNWPPWTFGQGTKVEVT

SEQ ID NO: 205 - protein, 127 aa, Homo sapiens
QVQLVQSGAEVKKPGASVKVSCKASGYNFISYGISWVRQAPGQGLEWMGWISAYNGNTNYAQKLQGRVTMTTDTSTSTAYMELRSLRSDDTAVYYCARVRIFGVVIIVDTAMVLDYWGQGTLVTVSS

SEQ ID NO: 206 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRLTFGGGTKVEIK

SEQ ID NO: 207 - protein, 127 aa, Homo sapiens
QVQLVQSGAEVKKPGSSVKVSCKASGGTFSSYAISWVRQAPGQGLEWMGRIIPILDITNYAQKFQGRVTITADKSTSTAYMELSSLRSEDTAVYYCARGLWRSSIAGLHVPYGMDVWGQGTTVTVSS

SEQ ID NO: 208 - protein, 110 aa, Homo sapiens
QSVLTQPPSVSAAPGQKVTISCSGSSSNIGNNYVSWYQQLPGTAPQLLIYDNNKRPSRIPDRFSGSKSGTSATLGITGLQTGDEAVYYCGTWDSSLSAVVFGGGTKLTVL

SEQ ID NO: 209 - protein, 122 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATGPTVTTVTNWFDPWGQGTLVTVSS

SEQ ID NO: 210 - protein, 110 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAEVFGTGTKVTVL

SEQ ID NO: 211 - protein, 127 aa, Homo sapiens; identical to SEQ ID NO: 205

SEQ ID NO: 212 - protein, 110 aa, Homo sapiens
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAGVFGGGTKLTVL

SEQ ID NO: 213 - protein, 119 aa, Homo sapiens
QVQLQQWGAGLLKPSESLSLAFFVYGGSFSGYYWSWIRQPPGKGLEWIGEINHSGSTNYNPSLKSRVTISVDTSKNQFSLKLSSVTAADTAVYYCARGGGYSYGAIDYWGQGTLVTVSS

SEQ ID NO: 214 - protein, 108 aa, Homo sapiens
QSVLTQPPSASGTPGQRVTISCSGSSSNIGTNYVYWYQQLPGTAPKLLIYRNTQRPSGVPDRFSGSKSGTSASLAISGLRSEDEADYYCASWDDSHVVFGGGTKLTVL

SEQ ID NO: 215 - protein, 128 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARVPRPPWDDFWSGYLYYFDYWGQGTLVTVSS

SEQ ID NO: 216 - protein, 109 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPPGFTFGPGTKVDIK

SEQ ID NO: 217 - protein, 120 aa, Homo sapiens
QVQLVESGGGVVQPGRSLRLSCAASGFTFSSYAMHWVRQAPGKGLEWVAVISYDGDNKYYADSVKGRFTISRDNSKNTLYLQMNSLRAEDTAVYYCARDPTAVTGPFDYWGQGTLVTVSS

SEQ ID NO: 218 - protein, 106 aa, Homo sapiens
SYELTQPLSVSVALGQTARITCGGNNIGSKNVHWYQQKPGQAPVLVIYRDSNRPSGIPERFSGSNSGNTATLTISRAQAGDEADYFCQVWDSSTVVFGGGTKLTVL

SEQ ID NO: 219 - protein, 126 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARLSGIFGVVIIPGFFDYWGQGTLVTVSS

SEQ ID NO: 220 - protein, 108 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSYSLLTFGGGTKVEIK

SEQ ID NO: 221 - protein, 122 aa, Homo sapiens
QVQLQESGPGLVKPSETLSLTCTVSGGSISSYYWSWIRQPAGKGLEWIGRIYTSGSTNYNPSLKSRVTMSVDTSKNQFSLKLSSVTAADTAVYYCAREDSYGSGSTGAFDIWGQGTMVTVSS

SEQ ID NO: 222 - protein, 107 aa, Homo sapiens
SYELTQPPSVSVSPGQTARITCSGDALPKQYAYWYQQKPGQAPVLVIYKDSERPSGIPERFSGSSSGTTVTLTISGVQAEDEADYYCQSADSSGTYVFGTGTKVTVL

SEQ ID NO: 223 - protein, 127 aa, Homo sapiens
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTRVHHCSGGSCYGYYFDYWGQGTLVTVSS

SEQ ID NO: 224 - protein, 106 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWVTFGGGTKVEIK

SEQ ID NO: 225 - protein, 125 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTYYDFWSGSTNYYGMDVWGQGTTVTVSS

SEQ ID NO: 226 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKLEIK

SEQ ID NO: 227 - protein, 130 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHIAGLEWLFRAAAVLNWFDPWGQGTLVTVSS

SEQ ID NO: 228 - protein, 108 aa, Homo sapiens
  misc_feature (91)..(91): Xaa can be any naturally occurring amino acid
EIVLTQSPGTLSLSPGKRATLSCRASQSVSSSYLAWYQQKPGQAPRLLIYGASSRATGIPDRFSGSGSGTDFTLTISRLEPEDFAVYYCQXYGSSPNTFGQGTKLEIK

SEQ ID NO: 229 - protein, 123 aa, Homo sapiens
EVQLLESGGGLVQPGGSLKLSCAASGFTFNSYAMTWVRQAPGKGLEWLSTISGSGGRTYYADSVKGRFAISRDNSKNTVFLQMSTLRAEDTAVYFCARDFYGLESFYPGIDSWGQGTLVTVSS

SEQ ID NO: 230 - protein, 107 aa, Homo sapiens
SYELTQPSSVSVFPGQTARITCSGDVLEKKYARWFQQKPGQAPLLVIYKDSERPSGIPERFFGSSSGTTVTLTISGAQVEDEADYYCYSAADNNRGVFGGGTKLTVL

SEQ ID NO: 231 - protein, 122 aa, Homo sapiens
QVQLVQSGAEVKKPGSSVKVSCKASGGTFSSYAITWVRQAPGQGLEWMGGIIPIFGTANYAQKFQGRLTITADDSTSTAYMELSSLKSEDTAVYYCARFRAYDSSGYGPDYWGQGTLVTVSS

SEQ ID NO: 232 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSITFGQGTRLEIK

SEQ ID NO: 233 - protein, 133 aa, Homo sapiens
EVQLLESGGGLGQPGGSLRLSCAASGFTFSNYAMSWVRQAPGKGLEWVSVISGSGGSTYYADSVKGRFTISRDNSKNTLYLQMNSLRAEDTAVYYCAKAGAVGYYDSNGYYYDVDYYYGMDVWGQGTTVTVSS

SEQ ID NO: 234 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVL

SEQ ID NO: 235 - protein, 120 aa, Homo sapiens
EVQLVESGGGLVQPGGSLRLSCAASGFTVSTNYMSWVRQAPGKGLEWVSVIYSGGSTYYVDSVKGRFTISRDNSKNTLYLQMNSLRAEDTAVYYCASTNSGSYSHVFDIWGQGTMVTVSS

SEQ ID NO: 236 - protein, 106 aa, Homo sapiens
DIQMTQSPSSLSASVGDRVTITCQASQDISNYLNWYQQKPGKAPKLLIYDASNLKTGVPSRFSGSGSGTDFTFTISSLQPEDIATYYCQQYDNLLTFGGGTKVEIK

SEQ ID NO: 237 - protein, 125 aa, Homo sapiens
QVQLQESGPGLVKPSETLSLTCTVSGGSVSSGSYYWSWIRQPPGKGLEWIGYIYYSGSTNYKYSLKSRVTISVDTSKNQFSLKLSSVTAADTAVYYCAREKGGTEWEFNDAFDIWGQGTMVTVSS

SEQ ID NO: 238 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSAPRTFGQGTKVEIK

SEQ ID NO: 239 - protein, 121 aa, Homo sapiens
EVQLVQSGAEVKKPGESLRISCKGSGYSFTSYWISWVRQMPGKGLEWMGRIDPSDSYTNYSPSFQGHVTISADKSISTASLQWSSLKASDTAMYYCARQKRDGTTWAFDYWGQGTLVTASS

SEQ ID NO: 240 - protein, 107 aa, Homo sapiens
DIQMTQSPSSLSASVGDRVTITCQASQDISNYLNWYQQKPGKAPKLLIYDASNLETGVPLRFSGSGSGTDFTFTISSLQPEDIATYYCQQYDNLPLTFGGGTKVEIK

SEQ ID NO: 241 - protein, 121 aa, Homo sapiens
QVQLQESGPGLVKPSETLSLTCTVSGGSISSYYWSWIRQPPGKGLEWIGYVYYSGSTNYNPSLKSRVTISVDTSKNEFSLKLSSVTAADTAVYYCASSQRPDGNLYYFDYWGQGTLVTVSS

SEQ ID NO: 242 - protein, 108 aa, Homo sapiens
SYVLTQPPSVSVAPGQTARITCGGNNIGSKTVHWYQQKPGQAPVLVVYDDSDRPSGIPERFSGSNSGNTATLTISRVEAGDEADYYCQVWDSSSDHYVFGTGTKVTVL

(19) United States
(12) Patent Application Publication
(10) Pub. No.: US 2023/0212266 A1
(43) Pub. Date: Jul. 6, 2023

(54) NEUTRALIZING MONOCLONAL ANTIBODIES AGAINST COVID19

(71) Applicant: Fred Hutchinson Cancer Center, Seattle, WA (US)

(72) Inventors: Leonidas Stamatatos, Seattle, WA (US); Andrew McGuire, Seattle, WA (US); Marie Pancera, Seattle, WA (US)

(73) Assignee: Fred Hutchinson Cancer Center, Seattle, WA (US)

(21) Appl. No.: 17/997,093

(22) PCT Filed: Apr. 26, 2021
(86) PCT No.: PCT/US21/29216

§ 371 Date: Oct. 25, 2022

Related U.S. Application Data
(60) Provisional application No. 63/016,268, filed on Apr. 27, 2020.
(60) Provisional application No. 63/131,599, filed on Dec. 29, 2020.

(51) Int. Cl. C07K 16/10; A61P 11/00; A61P 31/14; A61K 45/06; A61K 39/42

(52) U.S. Cl. CPC C07K 16/10; A61P 11/00; A61P 31/14; A61K 45/06; A61K 39/42; C07K 2317/76; C07K 2317/21

(57) ABSTRACT

The disclosure provides antibodies, antibody fragments, and antibody derivatives that bind to the viral envelope spike protein (S2P) of Severe Acute Respiratory Syndrome Coronavirus 2 (SARS-CoV-2), and related compositions, and methods. In some embodiments, the antibodies, antibody fragments, and antibody derivatives bind to an epitope of the receptor binding domain (RBD) of SARS-CoV-2.

### CROSS-REFERENCES TO RELATED APPLICATIONS

This application claims the benefit of U.S. Patent Application No. 63/016,268, filed Apr. 27, 2020, and U.S. Patent Application No. 63/131,599, filed Dec. 29, 2020, the disclosures of which are incorporated herein by reference in their entireties.

### STATEMENT REGARDING SEQUENCE LISTING

The sequence listing associated with this application is provided in text format in lieu of a paper copy and is hereby incorporated by reference into the specification. The name of the text file containing the sequence listing is FHTM174299 Seq_List_final_20210421_ST25.txt. The text file is 252 KB; was created on Apr. 21, 2021; and is being submitted via EFS-Web with the filing of the specification.

### FIELD OF THE INVENTION

This disclosure relates to severe acute respiratory syndrome coronavirus 2 (SARS-CoV-2) antibodies and methods for their use.

### BACKGROUND

The WHO declared the 2020 COVID-19 to be a global pandemic on Mar. 11, 2020. The infection is caused by SARS-CoV-2, a beta coronavirus, with 79.5% genome sequence identity to SARS-CoV. The immune response to this infection is not well-understood. It is also unclear which types of immune responses are required to prevent or control severity of infection.

High-resolution structures of the SARS-CoV-2 prefusion-stabilized spike (S) ectodomain revealed that it adopts multiple conformations with either one receptor-binding domain (RBD) in the “up” or “open” conformation or all RBDs in the “down” or “closed” conformation, similar to previous reports on both SARS-CoV S and MERS-CoV S. Like SARS-CoV, SARS-CoV-2 utilizes angiotensin-converting enzyme 2 (ACE2) as an entry receptor binding with nM affinity. Indeed, the S proteins of the two viruses share a high degree of amino acid sequence homology, 76% overall and 74% in RBD.

Although binding and neutralizing antibody responses are known to develop following SARS-CoV-2 infection, no information is currently available on the epitope specificities, clonality, binding affinities, and neutralizing potentials of the antibody response.

Accordingly, there remains a need for anti-SARS-CoV-2 monoclonal antibodies, such as neutralizing antibodies, that can assist the development of therapeutic interventions and screening tools, and can serve as templates for the development of an effective vaccine. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one embodiment, the disclosure provides an antibody or fragment or derivative thereof that binds to an epitope in the viral envelope spike protein (S2P) of a Severe Acute Respiratory Syndrome Coronavirus 2 (SARS-CoV-2). The antibody or fragment derivative thereof comprises the CDRs of a heavy chain variable domain (V_{H}), or the entire V_{H} sequence, comprising an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 (or a sequence with at least 90% identity thereto) and the CDRs of a light chain variable domain (V_{L}), or the entire V_{L} sequence, comprising an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242 (or a sequence with at least 90% identity thereto).

In some embodiments, the antibody is a monoclonal antibody or a fragment or derivative thereof. In some embodiments, the antibody or a derivative thereof is isolated. In some embodiments, the amino acid sequence of the heavy chain variable domain (V_{H}) is represented by SEQ ID NO: N and the amino acid sequence of the light chain variable domain (V_{L}) is represented by SEQ ID NO: N+1. In some embodiments, the monoclonal antibody or derivative thereof binds to an epitope of the receptor binding domain (RBD) of a Severe Acute Respiratory Syndrome Coronavirus 2 (SARS-CoV-2). In some embodiments, the monoclonal antibody or derivative thereof neutralizes SARS-CoV-2. In some embodiments, the monoclonal antibody or derivative thereof inhibits viral and cell membrane fusion. In some embodiments, the monoclonal antibody or derivative thereof is a fully human antibody.

In some embodiments, the monoclonal antibody or derivative thereof comprises a heavy chain variable domain (V_{H}) comprising amino acid sequence SEQ ID NO: 1 and a light chain variable domain (V_{L}) comprising amino acid sequence SEQ ID NO: 2.

In some embodiments, the antibody or derivative thereof comprises: a heavy chain variable domain (V_{H}) comprising amino acid sequence SEQ ID NO: 9 and a light chain variable domain (V_{L}) comprising amino acid sequence SEQ ID NO: 10; a heavy chain variable domain (V_{H}) comprising amino acid sequence SEQ ID NO: 55 and a light chain variable domain (V_{L}) comprising amino acid sequence SEQ ID NO: 56; a heavy chain variable domain (V_{H}) comprising amino acid sequence SEQ ID NO: 81 and a light chain variable domain (V_{L}) comprising amino acid sequence SEQ ID NO: 82; a heavy chain variable domain (V_{H}) comprising amino acid sequence SEQ ID NO: 109 and a light chain variable domain (V_{L}) comprising amino acid sequence SEQ ID NO: 110; a heavy chain variable domain (V_{H}) comprising amino acid sequence SEQ ID NO: 135 and a light chain variable domain (V_{L}) comprising amino acid sequence SEQ ID NO: 136; a heavy chain variable domain (V_{H}) comprising amino acid sequence SEQ ID NO: 175 and a light chain variable domain (V_{L}) comprising amino acid sequence SEQ ID NO: 176; a heavy chain variable domain (V_{H}) comprising amino acid sequence SEQ ID NO: 221 and a light chain variable domain (V_{L}) comprising amino acid sequence SEQ ID NO: 222; a heavy chain variable domain (V_{H}) comprising amino acid sequence SEQ ID NO: 223 and a light chain variable domain (V_{L}) comprising amino acid sequence SEQ ID NO: 224; a heavy chain variable domain (V_{H}) comprising amino acid sequence SEQ ID NO: 233 and a light chain variable domain (V_{L}) comprising amino acid sequence SEQ ID NO: 234; or a heavy chain variable domain (V_{H}) comprising amino acid sequence SEQ ID NO: 241 and a light chain variable domain (V_{L}) comprising amino acid sequence SEQ ID NO: 242.

In another aspect, the disclosure provides a composition comprising the antibody or fragment or derivative thereof, as described herein, and optionally a carrier.

In another aspect, the disclosure provides a method of preventing or treating a disease or disorder caused by a severe acute respiratory syndrome coronavirus 2 (SARS-CoV-2), comprising administering to a subject suffering from, or at risk of suffering from, the disease or disorder a therapeutically effective amount of the antibody or fragment or derivative thereof, or the composition, as described herein.

In some embodiments, the method further comprises administering an anti-viral drug, a viral entry inhibitor, or a viral attachment inhibitor.

In some embodiments, the antibody or derivative thereof is administered prior to or after exposure to SARS-CoV-2.

In another aspect, the disclosure provides a method of detecting the presence of a severe acute respiratory syndrome coronavirus 2 (SARS-CoV-2) in a sample, the method comprising contacting the sample with an antibody or fragment or derivative thereof as described herein and detecting the presence or absence of an antibody-antigen complex, thereby detecting the presence of a SARS CoV 2.

In some embodiments, the sample is obtained from blood, cheek scraping or swab, nasal swab, saliva, biopsy, urine, feces, sputum, nasal aspiration, or semen. In some embodiments, the sample is obtained from blood.

In another aspect, the disclosure provides a method of delaying the onset of one or more symptoms of severe acute respiratory syndrome coronavirus 2 (SARS-CoV-2) comprising administering to a subject at risk a therapeutically effective amount of the antibody or fragment or derivative thereof, or the composition, as described herein.

In another aspect, the disclosure provides a nucleic acid comprising a sequence encoding an amino acid sequence of any one of SEQ ID NOS 1-26, 29-38, 41-92, and 95-242.

In another aspect, the disclosure provides an antigen-binding composition comprising an antibody or antigen-binding fragment thereof that specifically binds to an epitope in the viral envelope spike protein (S2P) of a Severe Acute Respiratory Syndrome Coronavirus 2 (SARS-CoV-2), wherein the antibody or antigen-binding fragment or derivative thereof comprises a heavy chain variable domain (V_{H}) comprising an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 and a light chain variable domain (V_{L}) comprising an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242.

In some embodiments, the antibody or antigen-binding fragment thereof is a monoclonal antibody or a fragment thereof. In some embodiments, the amino acid sequence of the heavy chain variable domain (V_{H}) is represented by SEQ ID NO: N and the amino acid sequence of the light chain variable domain (V_{L}) is represented by SEQ ID NO: N+1. In some embodiments, the composition comprises two or more of the antibodies or antigen-binding antibody fragments.

In another aspect, the disclosure provides a diagnostic kit for detecting infection of a subject by severe acute respiratory syndrome coronavirus 2 (SARS-CoV-2), comprising at least one antibody or antibody fragment or derivative, as described herein. In some embodiments, the at least one antibody or antibody fragment or derivative is bound to a detectable labelling group.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C illustrate the purification of S2P and RBD from SARS-Cov-2. 1A and 1B are size exclusion chromatography traces of S2P and RBD, respectively. 1C is an SDS page under non-reducing (lines 1 and 3) and reducing (lines 2 and 4) conditions of S2P (lanes 1 and 2) and RBD (lanes 3 and 4).

FIGS. 2A and 2B illustrate the serum antibody reactivity to S2P (2A) and RBD (2B) from SARS-CoV-2. “COVID19(⁺)”: sera from three confirmed patients with SARS-CoV-2. “E_COV_1-8, 10”: nine sera from person infected with endemic CoV viruses. “Negative Control”: two sera from healthy individuals. The graphs represent total antibody response to S2P (2A) and RBD (2B), i.e., IgG, IgM, and IgA.

FIGS. 3A and 3B graphically illustrate results of a SARS CoV-2 pseudovirus neutralization assay. 3A illustrates entry of SAR CoV-2 pseudotyped HIV-1 into 293T cells expressing ACE2 are inhibited by anti-ACE2 antibodies but not by an anti-EBV monoclonal antibody (AMMO1). 3B illustrates that plasma from COVID-19 positive donors, but not negative control plasma collected from a healthy donor prior to the pandemic, neutralize SARS CoV-2 pseudovirus infection of 293 cells expressing ACE2.

FIGS. 4A and 4B illustrate B cell-staining with S2P and RBD. Class switched (IgM−IgG+) B cells were stained with S2P labeled with Bv710 or PE and with RBD labeled with APC. Single, BV710+/PE+ Be cells (4A) were sorted into individual wells of a 96 well plate. Sorted cells were monitored for RBD binding using indexed sorting (4B).

FIGS. 5A-7R graphically illustrate binding of mAbs generated from isolated SARS-CoV-S2P-specific B cells to SARS-CoV-2 SPS and to SARS-CoV-2 RBD. Each graph shows the shift (nm) over time for each indicated mAb.

FIG. 8 graphically illustrates the percent neutralization of SARS CoV-2 pseudovirus for increasing concentrations of indicated antibody. CV1 and CV30 were serially diluted and tested for their ability to neutralize SARS-CoV-2 pseudovirus infection of 293T cells stably expressing ACE2. An ACE2-FC fusion and the anti-EBV mAb AMMO1 were included as positive and negative controls. Data points represent the mean and, error bars indicate the standard deviation of quadruplicate replicates. Data are representative of 6 independent experiments.

FIGS. 9A-13I graphically illustrate binding of mAbs generated from SARS-CoV-S2P-specific B cells isolated from additional subjects with COVID-19 to SARS-CoV-2 SPS and to SARS-CoV-2 RBD, as described in FIGS. 5A-7R, and to SARS-S2P. Each graph shows the shift (nm) over time for each indicated mAb.

FIG. 14 illustrates the percent neutralization of SARS Cov2 SARS CoV-2 pseudovirus for increasing concentrations of the indicated antibodies. Neutralizing activity of select mAbs were measured in a SARS-CoV-2 pseudovirus assay. The left panel illustrates dose response curves and the right panel illustrates interpolated concentration that reduces infectivity by half (IC₅₀).

### DETAILED DESCRIPTION

This disclosure is based on the inventors' recovery and analysis of antibodies that bind to epitopes on severe acute respiratory syndrome coronavirus 2 (SARS-CoV-2) virus, described in more detail below.

Antibody-Based Constructs

In one aspect the disclosure provides an antibody or fragment or derivative thereof that binds to an epitope in the viral envelope spike protein (S2P) of a Severe Acute Respiratory Syndrome Coronavirus 2 (SARS-CoV-2).

The term “antibody” is used herein in the broadest sense and encompasses various antibody structures derived from any antibody-producing mammal (e.g., mouse, rat, rabbit, and primate including human), and which specifically bind to an antigen of interest (e.g., S2P). An antibody derivative refers to a molecule that incorporates one or more antibodies or antibody fragments and may optionally incorporate alterations and/or additional elements. An antibody fragment specifically refers to an intact portion or subdomain of a source antibody that still retains antigen-binding capability. Often, antibody derivatives incorporate at least some additional modification in the structure of the antibody or fragment thereof, or in the presentation or configuration of the antibody or fragment thereof. Exemplary antibodies of the disclosure include polyclonal, monoclonal and recombinant antibodies. Exemplary antibodies or antibody derivatives of the disclosure also include multi-specific antibodies (e.g., bispecific antibodies); humanized antibodies; murine antibodies; chimeric, mouse-human, mouse-primate, primate-human, etc.

In some embodiments, the disclosed antibodies disclosed are monoclonal antibodies. Monoclonal antibodies can be produced using hybridoma methods (see, e.g., Kohler, B. and Milstein, C. (1975) Nature 256:495-497 or as modified by Buck, D. W., et al., In Vitro, 18:377-381(1982). In some embodiments, the antibody of interest can be sequenced, and the polynucleotide sequence may then be cloned into a vector for expression or propagation. The sequence encoding the antibody of interest can be maintained in the vector in a host cell, and the host cell can then be expanded and frozen for future use.

As indicated, an antibody fragment is a portion or subdomain derived from or related to a full-length antibody, preferably including the complementarity-determining regions (CDRs), antigen binding regions, or variable regions thereof, and antibody derivatives refer to further structural modification or combinations in the resulting molecule. Illustrative examples of antibody fragments or derivatives encompassed by the present disclosure include Fab, Fab′, F(ab)₂, F(ab′)₂ and Fv fragments, diabodies, single-chain antibody molecules, V_{H}H fragments, V_{NAR} fragments, multi-specific antibodies formed from antibody fragments, nanobodies and the like. For example, an exemplary single chain antibody derivative encompassed by the disclosure is a “single-chain Fv” or “scFv” antibody fragment, which comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Another exemplary single-chain antibody encompassed by the disclosure is a single-chain Fab fragment (scFab).

As indicated, antibodies can be further modified to created derivatives that suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin. Any of these antibodies, or fragments or derivatives thereof, are encompassed by the disclosure. In some exemplary embodiments, described in more detail below, the antibodies or at least the antigen-binding domain (e.g., CDRs or V_{H} and V_{L} domains) are fully human. Such fragments and derivatives are all encompassed by embodiments of the present disclosure.

Antibody fragments and derivatives that recognize specific epitopes can be generated by any technique known to those of skill in the art. For example, Fab and F(ab′)₂ fragments of the disclosure can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies, or fragments or derivatives thereof, of the present disclosure can also be generated using various phage display methods known in the art. Finally, the antibodies, or fragments or derivatives thereof, can be produced recombinantly according to known techniques.

It will be apparent to the skilled practitioner that the binding domain can comprise antigen binding molecules other than antibody-based domain, such as peptidobodies, antigen-binding scaffolds (e.g., DARPins, HEAT repeat proteins, ARM repeat proteins, tetratricopeptide repeat proteins, and other scaffolds based on naturally occurring repeat proteins, etc. [see, e.g., Boersma and Pluckthun, Curr. Opin. Biotechnol. 22:849-857, 2011, and references cited therein, incorporated herein by reference]), which include a functional binding domain or antigen-binding fragment thereof.

As used herein, the term “specifically bind” or variations thereof refer to the ability of the binding domain (e.g., of the antibody, or fragment or derivative thereof) to bind to the antigen of interest (e.g., S2P and/or RBD), without significant binding to other molecules, under standard conditions known in the art. The binding domain can bind to other peptides, polypeptides, or proteins, but with lower affinity as determined by, e.g., immunoassays, BIAcore, or other assays known in the art. However, the binding domain preferably does not substantially cross-react with other antigens.

In some embodiments, the binding domain (e.g., domain binding to S2P and/or RBD) of the antibody, or fragment or derivative thereof has a binding affinity within a range characterized by a dissociation constant (K_{d}) from about 50 nM (lower binding affinity) to about 0.001 nM (higher binding affinity). For example, the binding domain has a binding affinity for the antigen (e.g., S2P and/or RBD) characterized by (K_{d}) of about 50 nM, 40 nM, 30 nM, 20 nM, 10 nM, 5 nM, 1 nM, 0.75 nM, 0.5 nM, 0.1 nM, 0.05 nM, 0.01 nM, 0.005 nM, and 0.001 nM, or even smaller. Typical (K_{d}) ranges characterizing the binding affinity of the cell-targeting domain for the antigen characteristic of the cell-type of interest include from about 30 nM to about 10 nM, from about 20 nM to about 1 nM, from about 10 nM to about 0.1 nM, from about 0.5 nM to about 0.05 nM, and from about 0.1 nM to about 0.001 nM, or even lower, or any subrange therein.

In some embodiments, the antibody or fragment or derivative thereof, comprises the heavy and light chain complementarity determining regions (CDRs) of a heavy chain variable domain (V_{H}) and light chain variable domain (V_{L}) pair, such as pairs wherein the V_{H} comprises an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 and the V_{L} comprises an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242. In some embodiments, the antibody or fragment or derivative thereof, comprises the heavy and light chain complementarity determining regions (CDRs) of a heavy chain variable domain (V_{H}) and light chain variable domain (V_{L}) pair, wherein the amino acid sequence of the heavy chain variable domain (V_{H}) from the above list is represented by SEQ ID NO: N and the amino acid sequence of the light chain variable domain (V_{L}) from the above list is represented by SEQ ID NO: N+1. For example, the paired V_{H} and V_{L} domain sequences that provide the combination of CDRs comprise the following respective amino acid sequences SEQ ID NOS: 1 and 2 (CV1), 3 and 4 (CV2), 5 and 6 (CV3), 7 and 8 (CV4), 9 and 10 (CV5), 11 and 12 (CV6), 13 and 14 (CV7), 15 and 16 (CV8), 17 and 18 (CV9), 19 and 20 (CV10), 21 and 22 (CV11), 23 and 24 (CV12), 25 and 26 (CV13), 29 and 30 (CV15), 31 and 32 (CV116), 33 and 34 (CV17), 35 and 36 (CV18), 37 and 38 (CV19), 41 and 42 (CV21), 43 and 44 (CV22), 45 and 46 (CV23), 47 and 48 (CV24), 49 and 50 (CV25), 51 and 52 (CV26), 53 and 54 (CV27), 55 and 56 (CV30), 57 and 58 (CV31), 59 and 60 (CV32), 61 and 62 (CV33), 63 and 64 (CV34), 65 and 66 (CV35), 67 and 68 (CV36), 69 and 70 (CV37), 71 and 72 (CV38), 73 and 74 (CV39), 75 and 76 (CV40), 77 and 78 (CV41), 79 and 80 (CV42), 81 and 82 (CV43), 83 and 84 (CV44), 85 and 86 (CV45), 87 and 88 (CV46), 89 and 90 (CV47), 91 and 92 (CV48), 95 and 96 (CV50), 97 and 98 (CV2-1), 99 and 100 (CV2-2), 101 and 102 (CV2-3), 103 and 104 (CV2-4), 105 and 106 (CV2-5), 107 and 108 (CV2-6), 109 and 110 (CV2-7), 111 and 112 (CV2-8), 113 and 114 (CV2-9), 115 and 116 (CV2-10), 117 and 118 (CV2-11), 119 and 120 (CV2-12), 121 and 122 (CV2-13), 123 and 124 (CV2-14), 125 and 126 (CV2-15), 127 and 128 (CV2-16), 129 and 130 (CV2-17), 131 and 132 (CV2-18), 133 and 134 (CV2-19), 135 and 136 (CV2-20), 137 and 138 (CV2-21), 139 and 140 (CV2-22), 141 and 142 (CV2-23), 143 and 144 (CV2-24), 145 and 146 (CV2-25), 147 and 148 (CV2-26), 149 and 150 (CV2-27), 151 and 15 (CV2-28)2, 153 and 154 (CV2-29), 155 and 156 (CV2-30), 157 and 158 (CV2-32), 159 and 160 (CV2-33), 161 and 162 (CV2-34), 163 and 164 (CV2-35), 165 and 166 (CV2-36), 167 and 168 (CV2-37), 169 and 170 (CV2-38), 171 and 172 (CV2-39), 173 and 174 (CV2-40), 175 and 176 (CV2-41), 177 and 178 (CV2-42), 179 and 180 (CV2-43), 181 and 182 (CV2-44), 183 and 184 (CV2-45), 185 and 186 (CV2-46), 187 and 188 (CV2-47), 189 and 190 (CV2-48), 191 and 192 (CV2-49), 193 and 194 (CV2-50), 195 and 196 (CV2-52), 197 and 198 (CV2-53), 199 and 200 (CV2-54), 201 and 202 (CV2-55), 203 and 204 (CV2-56), 205 and 206 (CV2-57), 207 and 208 (CV2-58), 209 and 210 (CV2-59), 211 and 212 (CV2-60), 213 and 214 (CV2-61), 215 and 216 (CV2-62)v, 217 and 218 (CV2-63), 219 and 220 (CV2-64), 221 and 222 (CV2-65), 223 and 224 (CV2-66), 225 and 226 (CV2-67), 227 and 228 (CV2-68), 229 and 230 (CV2-69), 231 and 232 (CV2-70), 233 and 234 (CV2-71), 235 and 236 (CV2-72), 237 and 238 (CV2-73), 239 and 240 (CV2-74), or 241 and 242 (CV2-75). The mAb clone names from which the pair of V_{H} and V_{L} domains, and thus the associated CDRs, were obtained are provided in parentheses in the above list. The clone names are reflected in various figures of the present disclosure. Methods for identifying CDRs from a variable domain (i.e., V_{H} and V_{L} domains) sequences are known and are applicable to establish the above CDRs from the disclosed sequences. Exemplary approaches applicable here include, e.g. the Kabat, Chothia, and IMGT numbering schemes. See, e.g., Dondelinger, M. et al. “Understanding the Significance and Implications of Antibody Numbering and Antigen-Binding Surface/Residue Definition.” Frontiers in Immunology. vol. 9:2278. 16 Oct. 2018, incorporated herein by reference in its entirety.

In some embodiments, the antibody or fragment or derivative thereof, a heavy chain variable domain (V_{H}) comprising an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241, or a sequence with at least 90% (e.g., 91%, 92%, 93%, 94%, 95%, 96%, 97%, 98%, or 99%) identity thereto, and a light chain variable domain (V_{L}) comprising an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242, or a sequence with at least 90% (e.g., 91%, 92%, 93%, 94%, 95%, 96%, 97%, 98%, or 99%) identity thereto. In some embodiments, the antibody or fragment or derivative thereof, comprises a heavy chain variable domain (V_{H}) and light chain variable domain (V_{L}) pair, wherein the amino acid sequence of the heavy chain variable domain (V_{H}) from the above list is represented by SEQ ID NO: N and the amino acid sequence of the light chain variable domain (V_{L}) from the above list is represented by SEQ ID NO: N+1. Exemplary pairings satisfying this element are listed above in the context of the V_{H} and V_{L} domains containing CDRs and are applicable in the present context but are not repeated. Furthermore, this disclosure encompasses embodiments of the above pairings but where the sequences have the above indicated sequence identities (e.g., a pairing of a V_{H} with a sequence having 94% sequence identity to SEQ ID NO: 1 and a V_{L} with a sequence having 96% sequence identity of SEQ ID NO: 2, etc.).

As indicated above, in some embodiments, the antibody or fragment or derivative thereof is a monoclonal antibody or is a fragment or derivative thereof. In some embodiments, the antibody or fragment or derivative thereof is isolated.

As indicated above, the antibody or fragment or derivative thereof binds to an epitope in the viral envelope spike protein (S) of a SARS-CoV-2 virus. In some embodiments, the antibody or fragment or derivative thereof binds to an epitope in the S2P protein, one of the ectodomain and which is the major antigenic determinant of the spike protein. Unless stated otherwise, reference herein to the S2P refers represents the entire ectodomain (e.g., residues 1-1208) of the S2P (GenBank: MN908947) (see Wrapp D, et al. Cryo-EM structure of the 2019-nCoV spike in the prefusion conformation. Science. 2020; 367(6483):1260-3; Pallesen J, et al. Immunogenicity and structures of a rationally designed prefusion MERS-CoV spike antigen. Proc Natl Acad Sci USA. 2017; 114(35):E7348-E57, each of which is incorporated herein by reference in its entirety).

In some embodiments, the antibody or fragment or derivative thereof neutralize SARS-CoV-2 upon binding. For example, in some embodiments, the antibody or fragment or derivative thereof can inhibit viral and cell membrane fusion, thereby preventing invasion of the virus into the cell.

In some embodiments, the antibody or fragment or derivative thereof also binds to the receptor binding domain (RBD) of a SARS-CoV-2 virus spike protein. As described in more detail below, the RBD represents only residues 319-591 of SARS-CoV-2 spike protein. As described below, the inventors discovered, clonally amplified, and characterized several antibody constructs that bind to the RBD of the S2P ectodomain. These were identified as mAb clones CV5, CV30, CV43 CV2-7, CV2-20, CV2-41, CV2-65, CV2-66, CV2-71, and CV2-75. Accordingly, in some embodiments, the antibody or fragment or derivative thereof comprises the heavy and light chain complementarity determining regions (CDRs) of a heavy chain variable domain (V_{H}) and light chain variable domain (V_{L}) pair selected from: a heavy chain variable domain (V_{H}) comprising amino acid sequence SEQ ID NO: 9 and a light chain variable domain (V_{L}) comprising amino acid sequence SEQ ID NO: 10; a heavy chain variable domain (V_{H}) comprising amino acid sequence SEQ ID NO: 55 and a light chain variable domain (V_{L}) comprising amino acid sequence SEQ ID NO: 56; a heavy chain variable domain (V_{H}) comprising amino acid sequence SEQ ID NO: 81 and a light chain variable domain (V_{L}) comprising amino acid sequence SEQ ID NO: 82; a heavy chain variable domain (V_{H}) comprising amino acid sequence SEQ ID NO: 109 and a light chain variable domain (V_{L}) comprising amino acid sequence SEQ ID NO: 110; a heavy chain variable domain (V_{H}) comprising amino acid sequence SEQ ID NO: 135 and a light chain variable domain (V_{L}) comprising amino acid sequence SEQ ID NO: 136; a heavy chain variable domain (V_{H}) comprising amino acid sequence SEQ ID NO: 175 and a light chain variable domain (V_{L}) comprising amino acid sequence SEQ ID NO: 176; a heavy chain variable domain (V_{H}) comprising amino acid sequence SEQ ID NO: 221 and a light chain variable domain (V_{L}) comprising amino acid sequence SEQ ID NO: 222; a heavy chain variable domain (V_{H}) comprising amino acid sequence SEQ ID NO: 223 and a light chain variable domain (V_{L}) comprising amino acid sequence SEQ ID NO: 224; a heavy chain variable domain (V_{H}) comprising amino acid sequence SEQ ID NO: 233 and a light chain variable domain (V_{L}) comprising amino acid sequence SEQ ID NO: 234; or a heavy chain variable domain (V_{H}) comprising amino acid sequence SEQ ID NO: 241 and a light chain variable domain (V_{L}) comprising amino acid sequence SEQ ID NO: 242. In further embodiments, the antibody or fragment or derivative thereof comprise the heavy chain variable domain (V_{H}) sequence and the light chain variable domain (V_{L}) sequence included in the pairings listed above. In yet other embodiments, the antibody or fragment or derivative thereof comprises a pair of a heavy chain variable domain (V_{H}) and a light chain variable domain (V_{L}) comprising sequences independently with at least 90% (e.g., 91%, 92%, 93%, 94%, 95%, 96%, 97%, 98%, or 99%) identity to the sequences of the pairings listed immediately above (e.g., a pairing of a V_{H} with a sequence having 94% sequence identity to SEQ ID NO: 9 and a V_{L} with a sequence having 96% sequence identity of SEQ ID NO: 10, etc.).

In some embodiments, the antibody or fragment or derivative thereof is detectably labeled. Many detectable labels are known that can be readily attached, covalently or non-covalently, to the antibody or fragment or derivative thereof and are encompassed by the disclosure.

In another aspect, the disclosure provides a composition that comprises two or more of the antibodies or antigen-binding antibody fragments or derivatives thereof, as described above.

Nucleic Acids and Related Constructs

In another aspect, the disclosure provides a nucleic acid molecule encoding any of the antibodies or fragments or derivatives thereof described herein, or components there. For example, the disclosure provides a nucleic acid comprising a sequence encoding an amino acid sequence of any one of SEQ ID NOS 1-26, 29-38, 41-92, and 95-242. Each sequence identifier with an odd number represents a V_{H} domain and each sequence identifier with an even number represents a V_{L}. In some embodiments, the nucleic acid comprises a sequence encoding a V_{H} domain with an amino acid sequence from the above list (i.e., with an odd sequence identifier number) and a sequence encoding a V_{L} with an amino acid sequence from the above list (i.e., with an even sequence identifier number). In some embodiments, the encoded V_{H} domain has an amino acid sequence represented by SEQ ID NO: N and the encoded V_{L} domain has an amino acid sequence represented by SEQ ID NO: N+1. Such representative pairings are described in more detail above in the context of the disclosed antibodies or fragments or derivatives thereof.

A person of ordinary skill in the art can use the genetic code to determine nucleic acid sequences that can encode antibodies or fragments or derivatives thereof based on the above disclosures. In some embodiments, the nucleic acid further comprises a promoter sequence operatively linked to the sequence encoding the antibodies or fragments or derivatives thereof. The term “promoter” refers to a regulatory nucleotide sequence that can activate transcription (expression) of a gene and/or splice variant isoforms thereof. A promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the encoding nucleic acid are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended person, as various promoters are known and commonly used in the art.

In some embodiments, the disclosure provides a vector comprising the nucleic acid described above. The vector can be any construct that facilitates the delivery of the nucleic acid to a target cell and/or expression of the nucleic acid within the cell. The vectors can be viral vectors, circular nucleic acid constructs (e.g., plasmids), or nanoparticles, and the like.

Various viral vectors are known in the art and are encompassed by the present disclosure. See, e.g., Machida, C. A. (ed.), Viral Vectors for Gene Therapy: Methods and Protocols, Humana Press, Totowa, N.J. (2003); Muzyczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno associated virus (AAV) vector, an adenovirus vector, a retrovirus vector, or a lentivirus vector. A specific embodiment of an AAV vector includes the AAV2.5 serotype.

Formulation and Administration

The disclosure also encompasses compositions that comprise the antibody or fragment or derivative thereof, described above. The compositions can be formulations appropriate for methods of administration for application to in vivo therapeutic settings in subjects (e.g., mammalian, e.g., human, subjects with COVID-19). According to skill and knowledge common in the art, the disclosed antibody or fragment or derivative thereof, encoding nucleic acids, and/or vectors comprising the nucleic acids, can be formulated with appropriate carriers, excipients, and/or non-active binders, and the like, for administration to target a SARS-CoV-2 virus.

Cells

In another aspect, the disclosure provides a cell comprising the nucleic acid encoding any antibody or fragment or derivative thereof, as described herein. In some embodiments, the cell comprises a vector, wherein the vector comprises the nucleic acid encoding any antibody or fragment or derivative thereof, as described herein. The cell is capable of expressing the any antibody or fragment or derivative thereof from the nucleic acid. For example, the nucleic acid and/or vector can be configured for expression of the any antibody or fragment or derivative thereof from the encoding nucleic acid within the cell. A promoter operatively linked to the nucleic acid can be appropriately configured to allow binding of the cell's RNA polymerase and one or more transcription factors to permit assembly of the transcriptional complex.

The disclosure encompasses any type of cell for this aspect.

While the disclosed cells can be useful for production or in vitro study of SARS-CoV-2 or related viruses, the disclosure also encompasses therapeutic compositions comprising the disclosed cells. The compositions can comprise appropriate culture media. In some embodiments, the compositions comprise appropriate carriers for in vivo administration.

Methods

In another aspect, the disclosure provides a method of preventing or treating a disease or disorder caused by a severe acute respiratory syndrome coronavirus 2 (SARS-CoV-2), comprising administering to a subject suffering from, or at risk of suffering from, the disease or disorder a therapeutically effective amount of the antibody or fragment or derivative thereof or a composition comprising the antibody or fragment or derivative thereof, as described herein. The methods also encompass administering a nucleic acid (e.g., in a vector) encoding the antibody or fragment or derivative thereof, as described herein, to the subject and permitting the expression of the antibody or fragment or derivative thereof in a target cell.

In some embodiments, the disease or disorder is designated COVID-19.

In some embodiments, the antibody or fragment or derivative thereof, or nucleic acid encoding the antibody or fragment or derivative thereof, can be administered in combination with one or more other antibodies of the disclosure, e.g. as a cocktail comprising more than one antibody. The antibody or fragment or derivative thereof, or nucleic acid encoding the antibody or fragment or derivative thereof, or a combination thereof can be administered at a dose sufficient to neutralize the SARS-CoV-2. In some embodiments, the method also includes administering an anti-viral drug, a viral entry inhibitor, or a viral attachment inhibitor. In some embodiments, the anti-viral drug is REMDESIVIR. In some embodiments, the antibody can be administered prior to or after exposure to SARS-CoV-2.

In another aspect, the disclosure provides a method of delaying the onset of one or more symptoms of severe acute respiratory syndrome coronavirus 2 (SARS-CoV-2)-caused infection (e.g., COVID-19). The method comprises administering to a person at risk of suffering from such infection a therapeutically effective amount of the antibody or fragment or derivative thereof or a composition comprising the antibody or fragment or derivative thereof, as described herein.

In another aspect, provided herein is a method of detecting the presence of a severe acute respiratory syndrome coronavirus 2 (SARS-CoV-2) in a sample. The method comprises contacting the sample with the antibody or fragment or derivative thereof of the disclosure, and detecting the presence or absence of an antibody-antigen complex, thereby detecting the presence of a SARS-CoV-2 in a sample. Any suitable sample can be used in the methods of the disclosure. In some embodiments, the sample can be obtained from blood, cheek scraping or swab, nasal swab, saliva, biopsy, urine, feces, sputum, nasal aspiration, or semen. In some embodiments, the sample is obtained from blood.

Kits

In another aspect, the disclosure provides kits comprising one or more antibodies of the disclosure or fragments or derivatives thereof. Kits include one or more containers comprising by way of example, and not limitation, one or more antibodies specific to severe acute respiratory syndrome coronavirus 2 (SARS-CoV-2) or combinations thereof or fragments thereof and instructions for use in accordance with any of the methods of the disclosure. In some embodiments of the kits, the antibodies are bound to a detectable label. Any suitable detectable label can be used, such as a fluorophore, a radioactive label, a colloidal gold particle, a magnetic particle, a quantum dot, etc.

Generally, instructions comprise a description of administration or instructions for performance of an assay. The containers can be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits are provided in suitable packaging. Suitable packaging includes, but is not limited to, vials, bottles, jars, flexible packaging (e.g., sealed Mylar or plastic bags), and the like. A kit can have a sterile access port (e.g. the container can be an intravenous solution bag or a vial having a stopper pierceable by a hypodermic injection needle). The container can also have a sterile access port (e.g. the container can be an intravenous solution bag or a vial having a stopper pierceable by a hypodermic injection needle). Kits can optionally provide additional components such as buffers and interpretive information. Normally, the kit comprises a container and a label or package insert(s) on or associated with the container.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having COVID-19. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., COVID-19), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., COVID-19), or any combination thereof. The term “therapeutic effect” refers to the reduction, elimination, or prevention of the disease or condition, symptoms of the disease or condition, or side effects of the disease or condition in the subject.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or noncanonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

This Example describes an in-depth characterization of the antibody responses to SARS-CoV-2 infection, including the clonality of the B cell responses. This work reveals whether and how the development of neutralizing antibody responses is linked with the severity of clinical symptoms and with the control of infection.

### Human Subject Characteristics and Available Biospecimens

B-cells and plasma were obtained from SARS-CoV-2-infected subjects from Seattle, Wash., USA, after positive diagnosis. Biospecimens were collected as follows: blood was collected in ACD tubes, processed. Plasma and viable peripheral blood mononuclear cells (PBMCs) were isolated and cryopreserved for future analysis. This study was IRB approved and all participants signed informed consent for use of biospecimens in biomedical research.

### Monitoring the Emergence, Evolution and Epitope Specificities of Binding and Neutralizing Serum Antibody Responses.

Expression and Purification of SARS-CoV-2 Envelope Glycoproteins

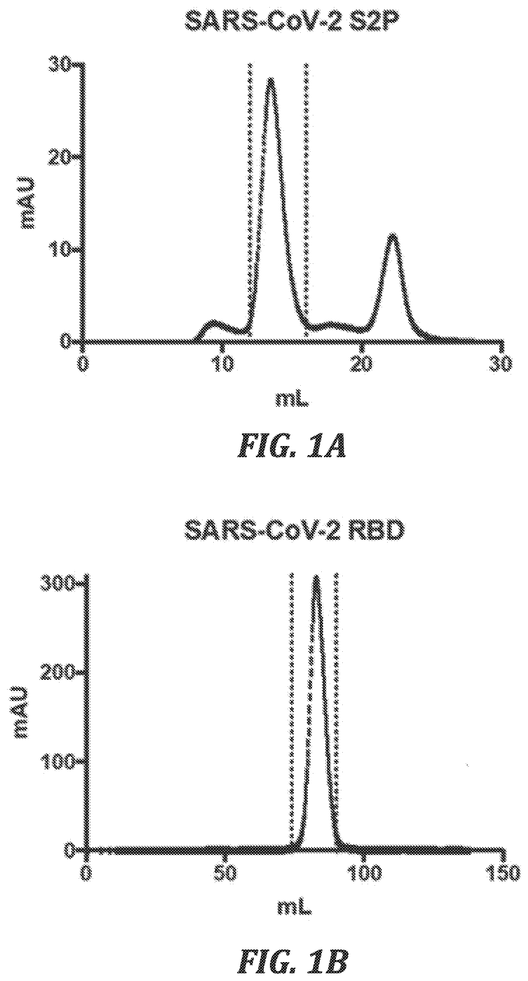

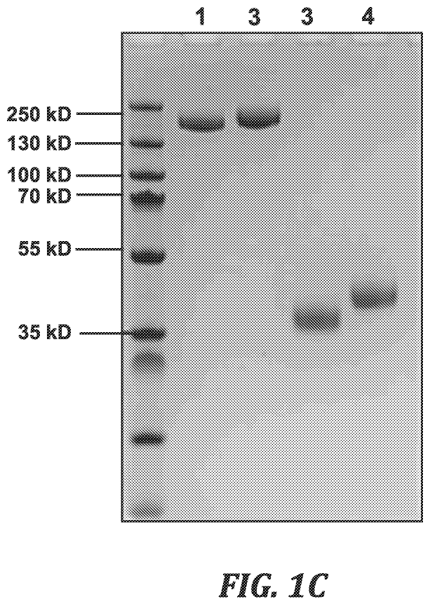

Coronavirus surface envelope glycoproteins are targets of neutralizing antibodies (Zhang H, et al. Identification of an antigenic determinant on the S2 domain of the severe acute respiratory syndrome coronavirus spike glycoprotein capable of inducing neutralizing antibodies. J Virol. 2004; 78(13):6938-45; Rockx B, et al. Structural basis for potent cross-neutralizing human monoclonal antibody protection against lethal human and zoonotic severe acute respiratory syndrome coronavirus challenge. J Virol. 2008; 82(7):3220-35; Wang L, et al. Importance of Neutralizing Monoclonal Antibodies Targeting Multiple Antigenic Sites on the Middle East Respiratory Syndrome Coronavirus Spike Glycoprotein To Avoid Neutralization Escape. J Virol. 2018; 92(10), and Walls A C, et al. Unexpected Receptor Functional Mimicry Elucidates Activation of Coronavirus Fusion. Cell. 2019; 176(5):1026-39 e15 each of which is incorporated herein by reference in its entirety). The SARS-CoV-2 envelope glycoprotein is a surface-exposed class-I fusion protein that, similarly to other coronaviruses, comprises an ectodomain, a transmembrane region and a short intracellular domain. The ectodomain consists of two non-covalently associated subunits: a receptor binding domain subunit (51) and a membrane-fusion subunit (S2). High resolution structures of SARS-CoV-2 envelope glycoproteins have been recently published (see Wrapp D, et al. Cryo-EM structure of the 2019-nCoV spike in the prefusion conformation. Science. 2020; 367(6483):1260-3; and Walls A C, et al. Structure, Function, and Antigenicity of the SARS-CoV-2 Spike Glycoprotein. Cell. 2020, each of which is incorporated herein by reference in its entirety). Vectors and expression platforms were used to produce large quantities of two different versions of the viral glycoprotein in 293 cells. Two forms of the SARS-CoV-2 envelope were expressed: S2P and receptor binding domain (RBD). The S2P represents the entire ectodomain and encodes residues 1-1208 (GenBank:MN908947) (Wrapp D, et al. Cryo-EM structure of the 2019-nCoV spike in the prefusion conformation. Science. 2020; 367(6483):1260-3; Pallesen J, et al. Immunogenicity and structures of a rationally designed prefusion MERS-CoV spike antigen. Proc Natl Acad Sci USA. 2017; 114(35):E7348-E57, each of which is incorporated herein by reference in its entirety). The RBD form encodes only residues 319-591 of SARS-CoV-2 S and is cloned upstream of a monomeric Fc separated by HRV3C protease cleavage site (Wrapp D, et al. Science. 2020; 367(6483):1260-3, incorporated herein by reference in its entirety). The vectors were obtained from Dr. McLellan who published the first atomic level structure of a stabilized prefusion SARS-CoV-2 Spike glycoproteins (Wrapp D, et al. Science. 2020; 367(6483):1260-3, incorporated herein by reference in its entirety). The two plasmids were transfected in 293 cells and the expressed proteins were purified using affinity purification followed by size exclusion chromatography (SEC) (FIGS. 1A-1C). Two mgs of S2P and 12 mgs of RBD were purified, which were employed for serological evaluation and B cell isolation (see below).

Serological Analysis

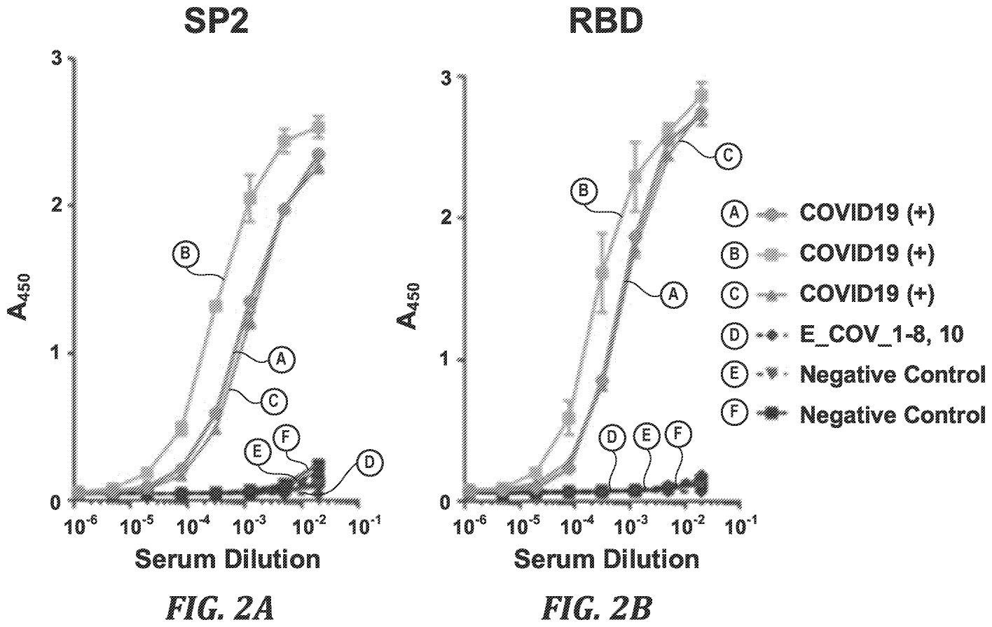

The two purified SARS-CoV-2 S proteins (S2P and RBD) were used in an ELISA assay with three SARS-CoV-2 seropositive serum samples (COVID-19(+)), two seronegative control sera (Negative Control) and sera from nine subjects infected with endemic CoV viruses in the Seattle metropolitan area (E_COV_1-8, 10) (FIGS. 2A and 2B). The sera from the three SARS-CoV-2 infected patients showed reactivity to both the S2P and the RBD. Neither the endemic CoV sera nor the negative control sera displayed reactivity to either SARS-CoV-2 proteins. These results indicate that the isolated viral proteins/peptides will specifically identify the presence of serum antibodies against the Env of SARS-CoV-2.

Neutralization Assays

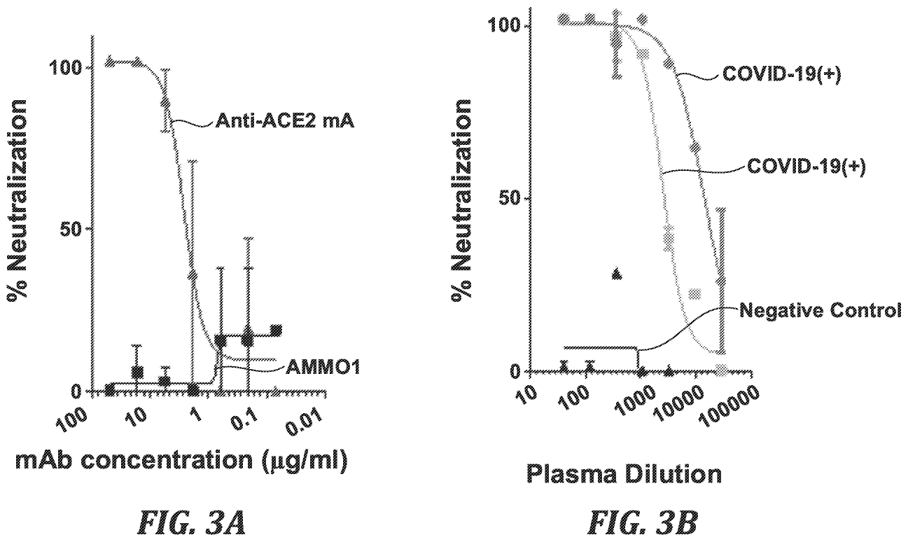

A pseudoviral neutralization assay was employed to assess the neutralizing activities of polyclonal sera and of mAbs derived from S2P- and RBD-specific B cells. Pseudoviral particles were produced in which the full-length S protein of SARS-CoV-2 were pseudotyped on an HIV backbone to deliver a luciferase reporter gene to target cells (293 cells expressing ACE2) upon entry. Consistent with a previous report, pseudoviral infection was inhibited by anti-ACE2 antibodies (Hoffmann M, et al. SARS-CoV-2 Cell Entry Depends on ACE2 and TMPRSS2 and Is Blocked by a Clinically Proven Protease Inhibitor. Cell. 2020, incorporated herein by reference in its entirety), but not a control anti-EBV control mAb (FIG. 3A) (Snijder J, et al. An Antibody Targeting the Fusion Machinery Neutralizes Dual-Tropic Infection and Defines a Site of Vulnerability on Epstein-Barr Virus. Immunity. 2018; 48(4):799-811 e9, incorporated herein by reference in its entirety). Plasma from donors with confirmed COVID-19 infection also neutralized SARS CoV-2 pseudovirus, whereas control plasma from an uninfected donor did not (FIG. 3B).

Isolation of Potent Neutralizing Monoclonal Antibodies from COVID19-Infected Patients

To identify the epitopes targeted by SARS-CoV-2 neutralizing antibodies, S2P- and RBD-specific B cells from the PBMCs of patients (once serum neutralizing activities were confirmed) were isolated. Using established methodologies (see Snijder J, et al. An Antibody Targeting the Fusion Machinery Neutralizes Dual-Tropic Infection and Defines a Site of Vulnerability on Epstein-Barr Virus. Immunity. 2018; 48(4):799-811 e9; Parks K R, et al. Overcoming Steric Restrictions of VRC01 HIV-1 Neutralizing Antibodies through Immunization. Cell reports. 2019; 29(10):3060-72 e7; and Dosenovic P, et al. Anti-idiotypic antibodies elicit anti-HIV-1-specific B cell responses. J Exp Med. 2019; 216(10):2316-30, each of which is incorporated herein by reference in its entirety), the V_{H} and V_{L} genes from individual B cells were sequenced and expressed as monoclonal antibodies (mAbs). The V_{H} and V_{L} sequence pairs are set forth in SEQ ID NOS:1-242. Their mAb binding (S2P, RBD- or dual-specific) affinities and neutralizing potencies were then determined, as described below.

Isolation of Antigen-Specific B Cells

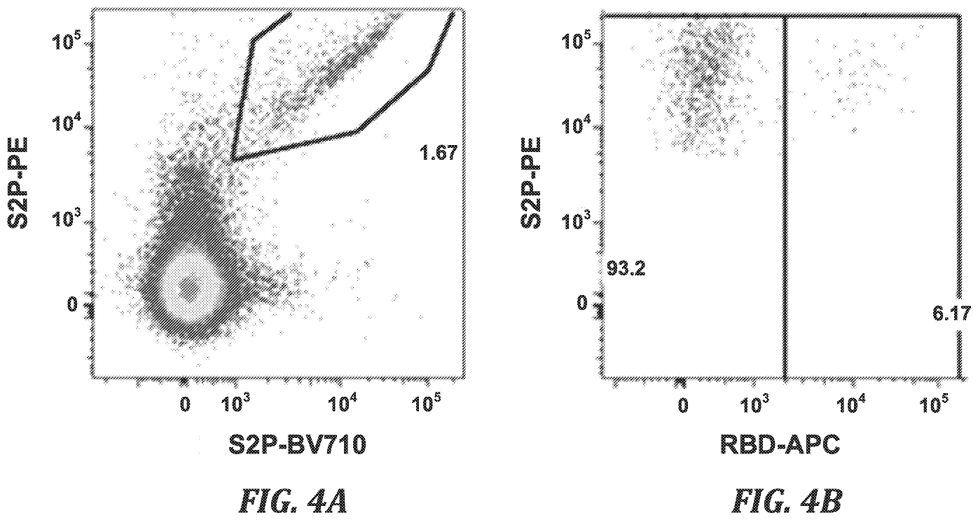

Initially, memory B cells from one SARS-CoV-2 patient (approximately 3 weeks post infection) were stained with S2P and RBD and single-cell sorted using standard B cell isolation protocols (FIGS. 4A and 4B). Here, IgG+ B cells were specifically sorted, first by staining the cells with S2P separately labeled with two different fluorophores and selecting the double positive cells (see gate, left panel). Within this population the cells that were specific for RBD, which was labeled with a different fluorophore, were identified (right panel). About 6% of the IgG+ B cells bound both RBD and S2P. In total, the 768 envelope-specific B cells were isolated and their respective nucleic acids encoding the V_{H} and V_{L} domains were sequenced. Incomplete V_{H} and V_{L} sequences were removed and V_{H}/V_{L} pairs were identified. Each V_{H}/V_{L} pair was expressed as IgG and tested for binding and neutralization. This was used as a pilot experiment to validate the reagents and staining protocols. Subsequently, the B cell isolation approach was performed from additional patients and the serum neutralizing activities were confirmed.

MAb-Binding

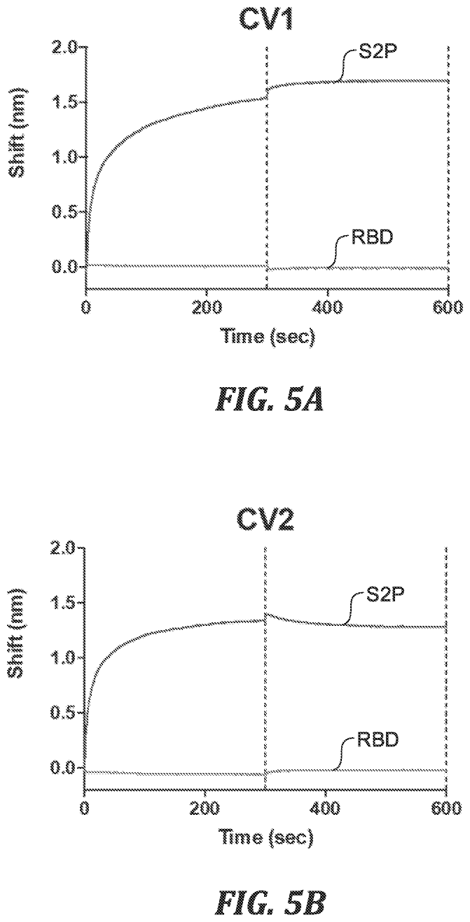

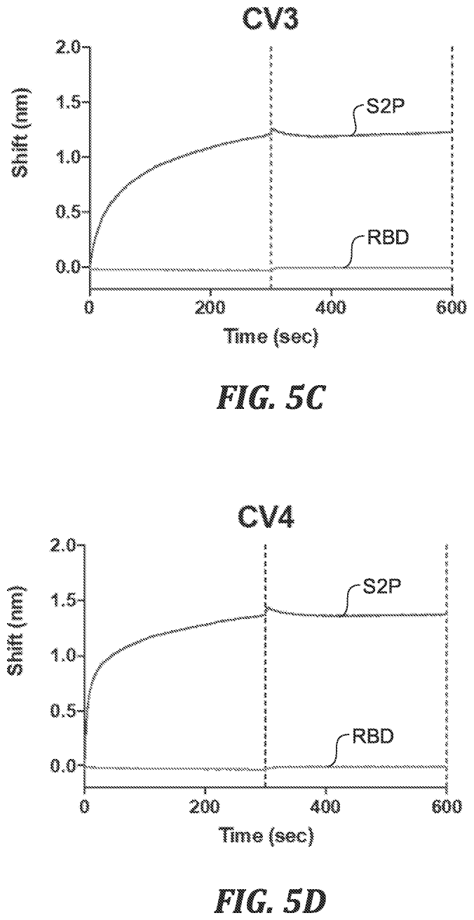

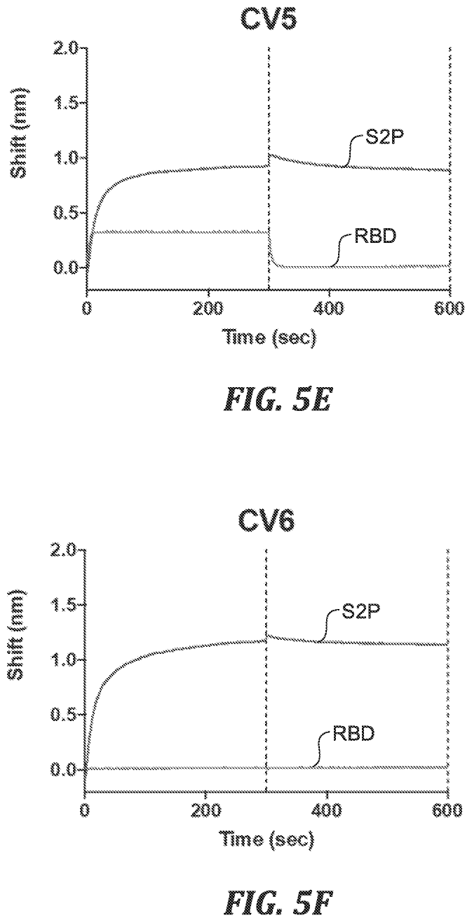

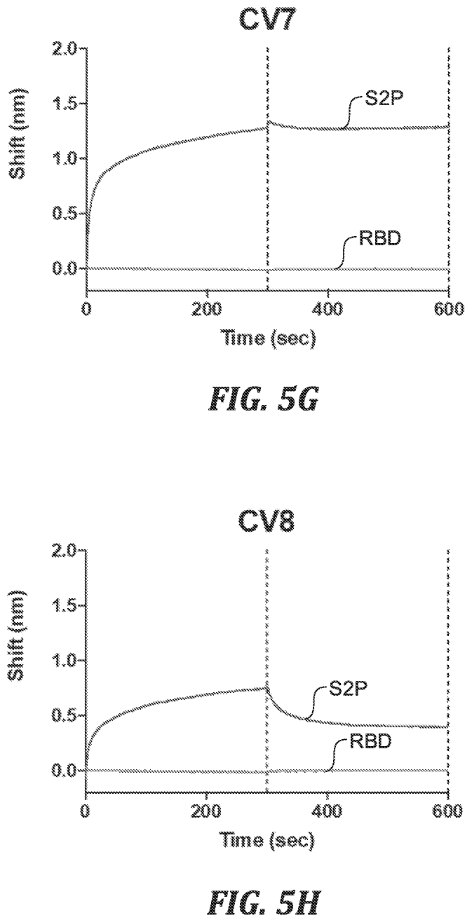

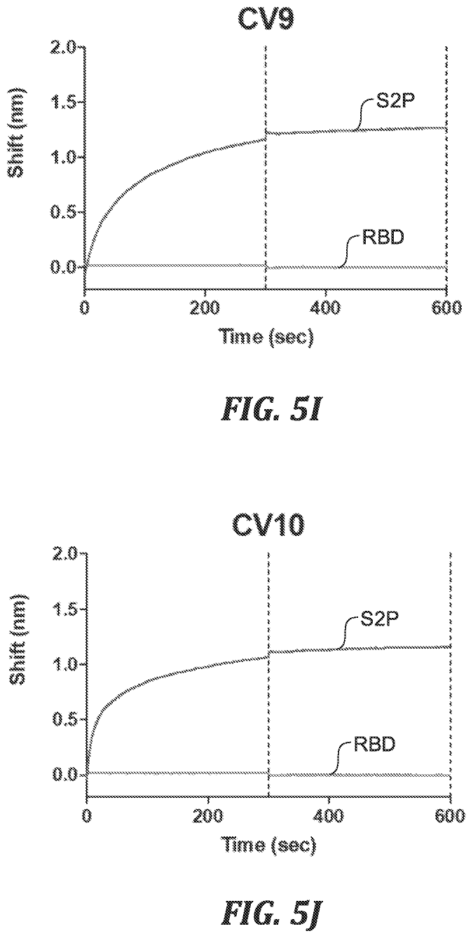

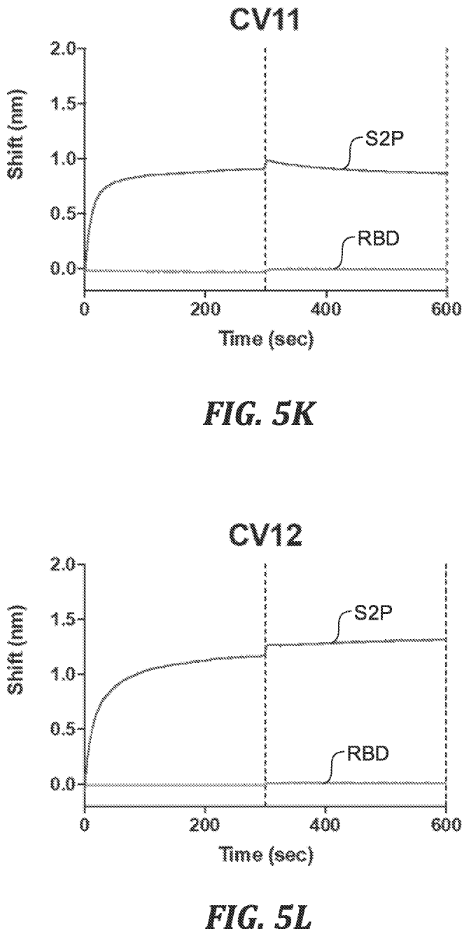

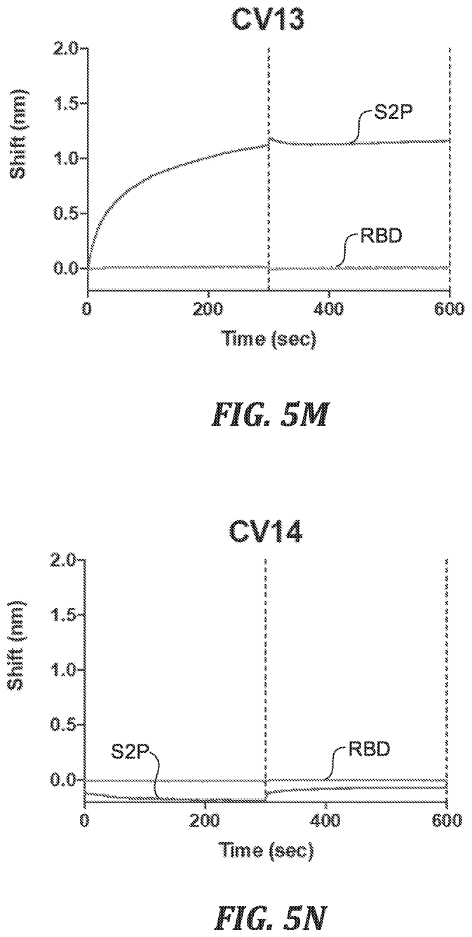

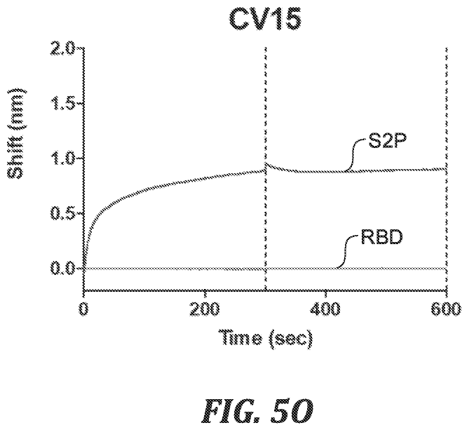

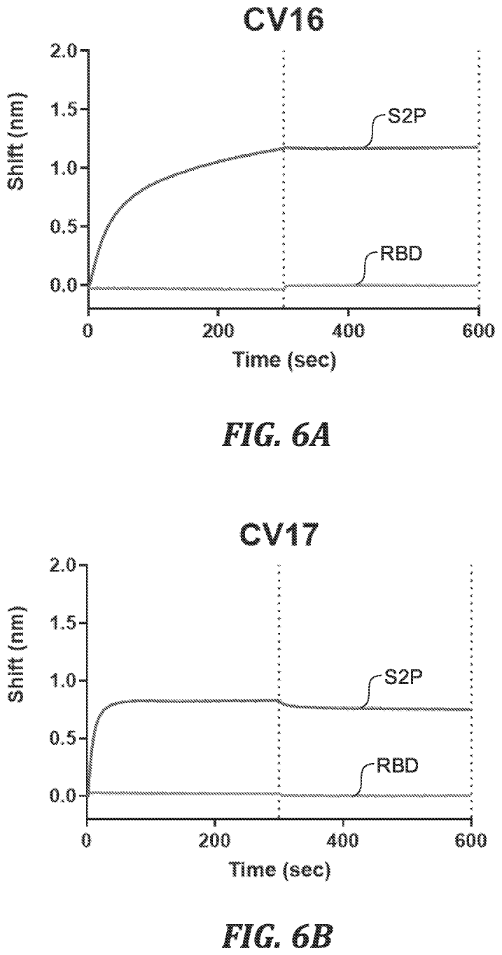

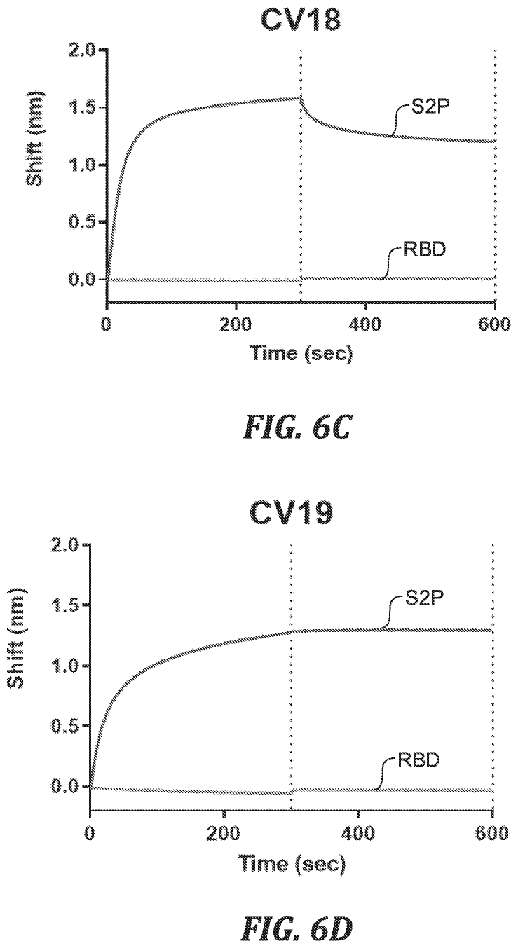

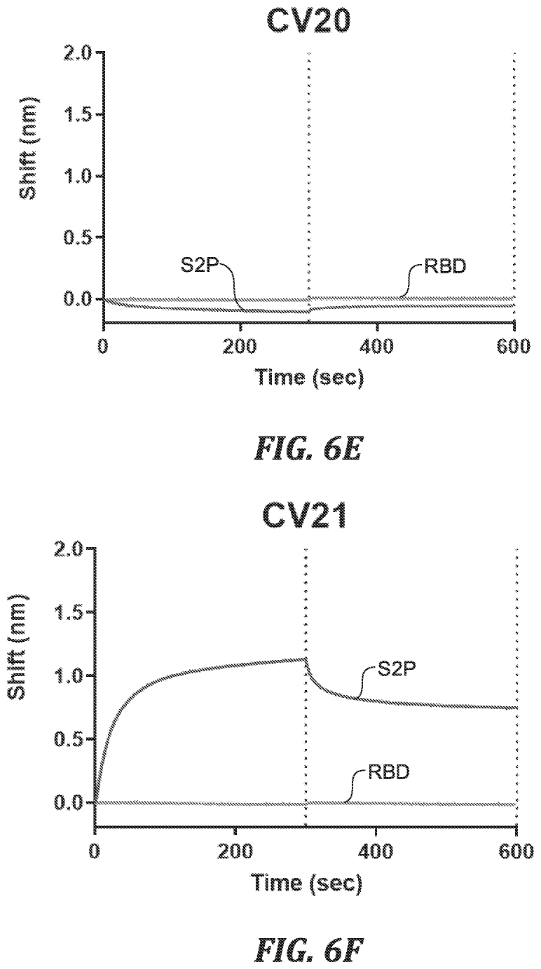

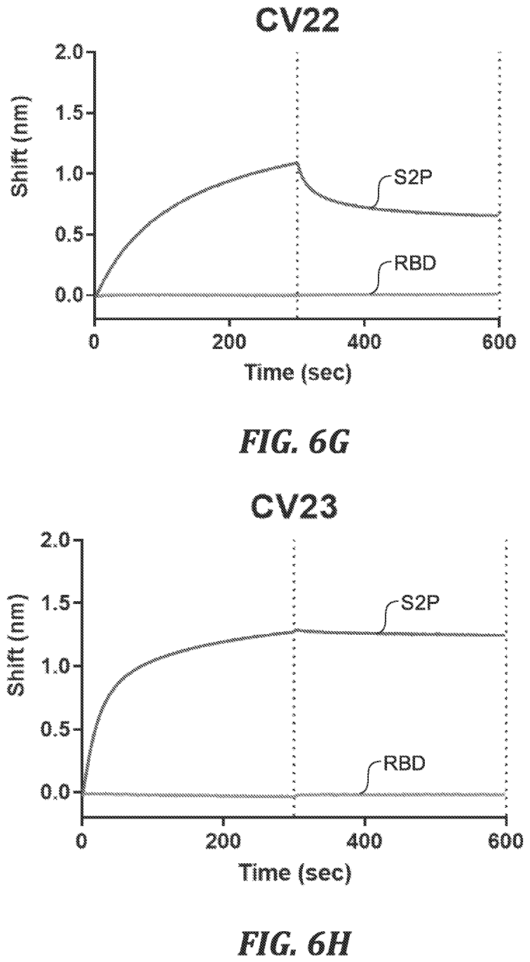

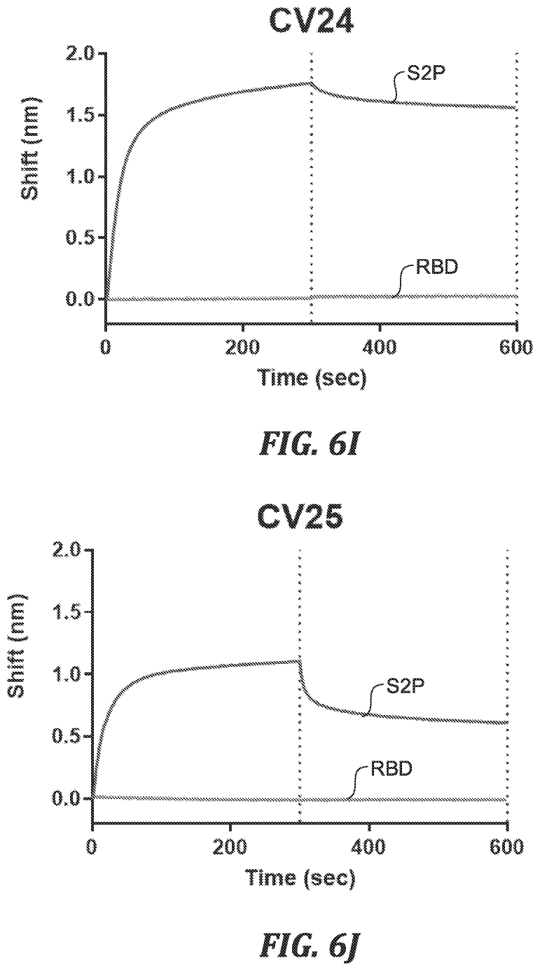

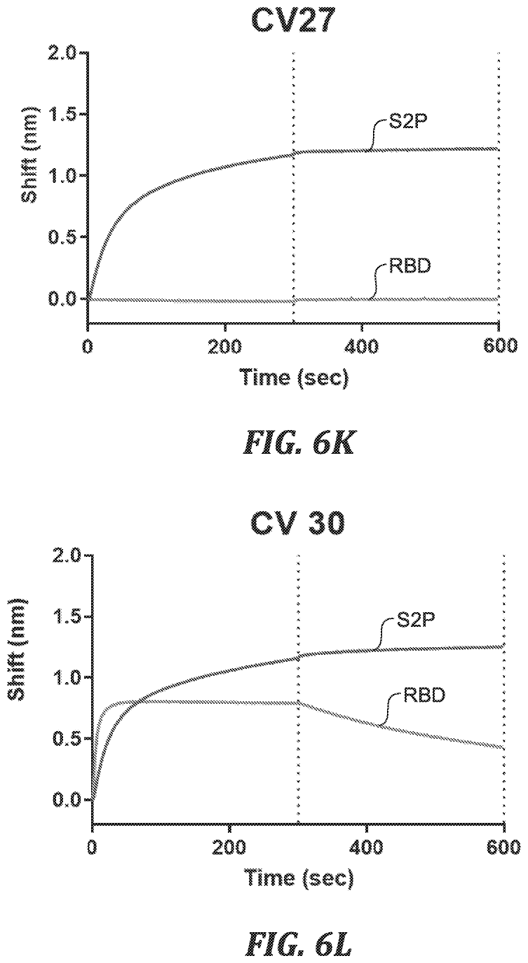

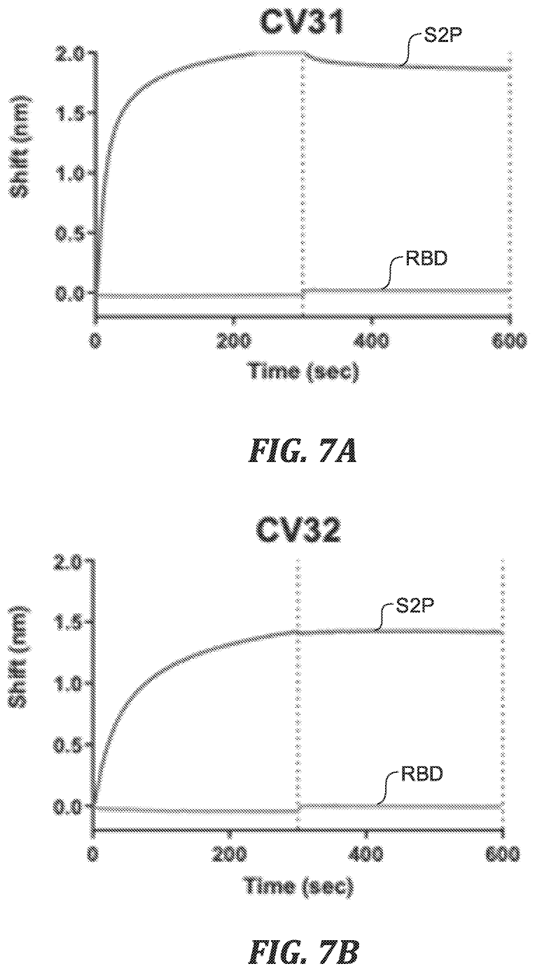

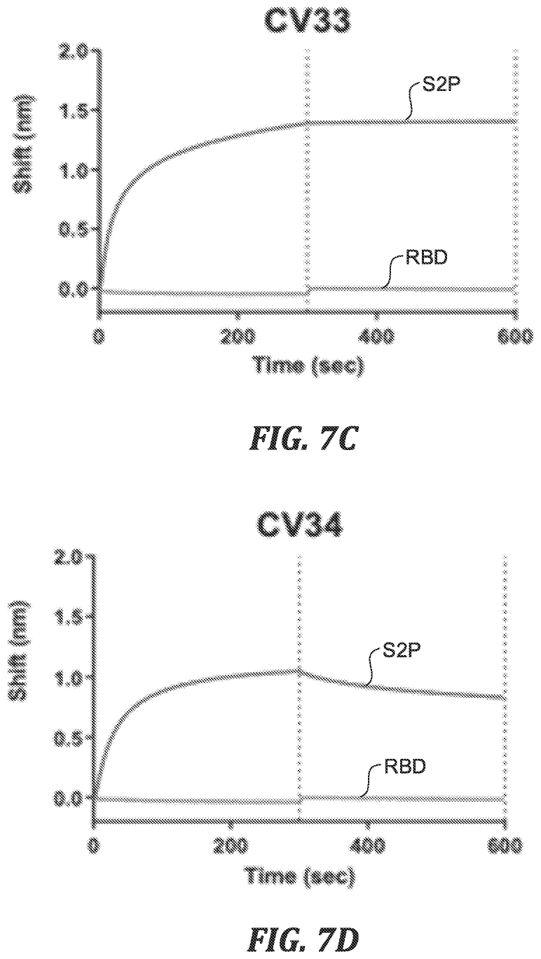

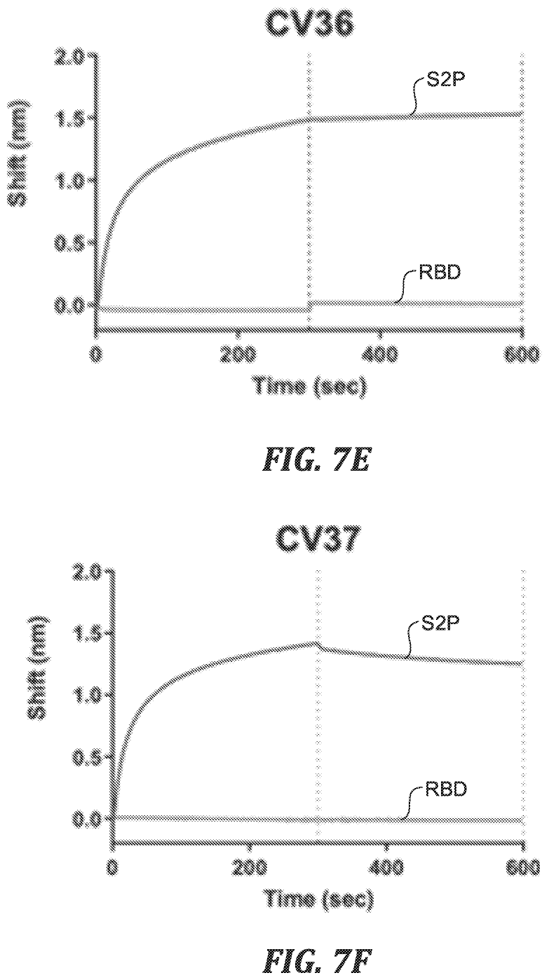

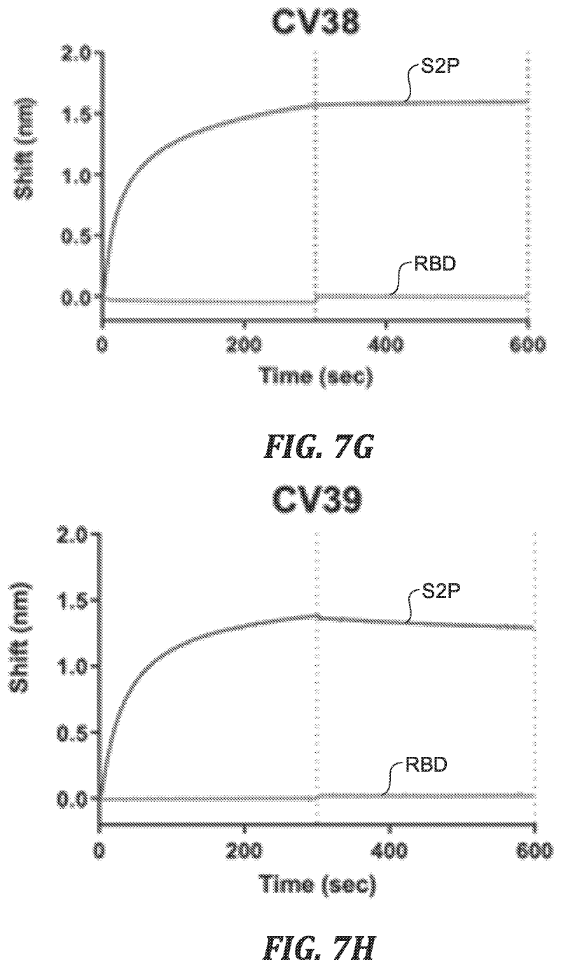

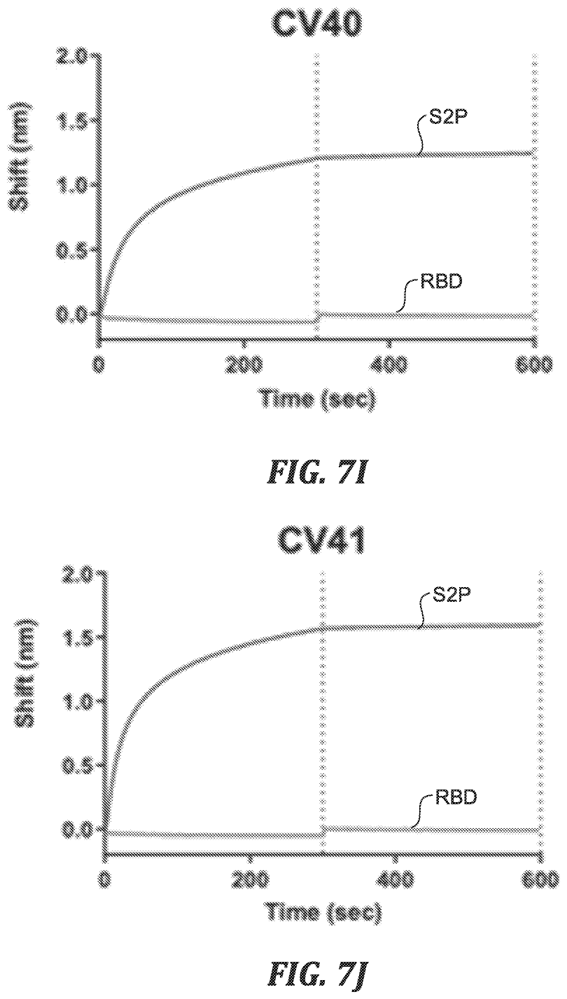

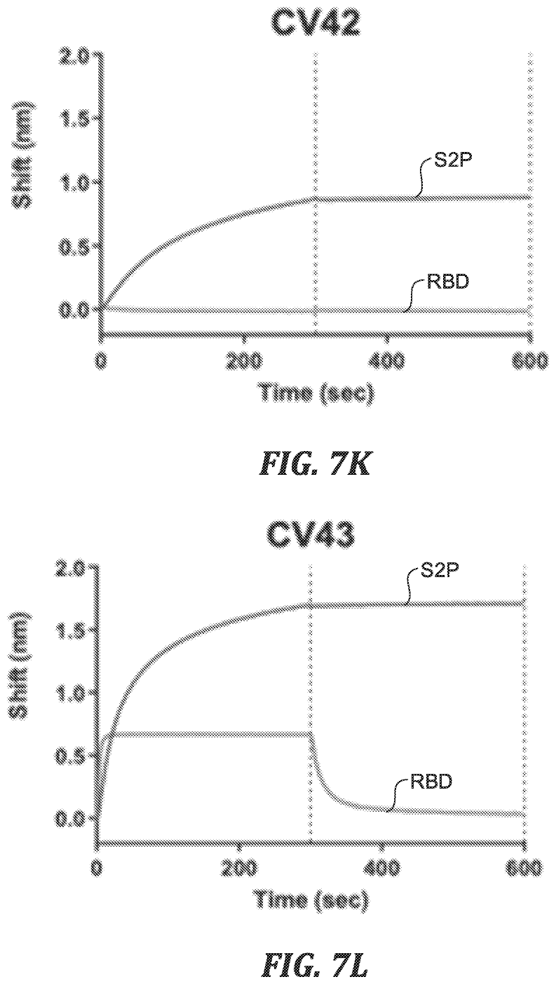

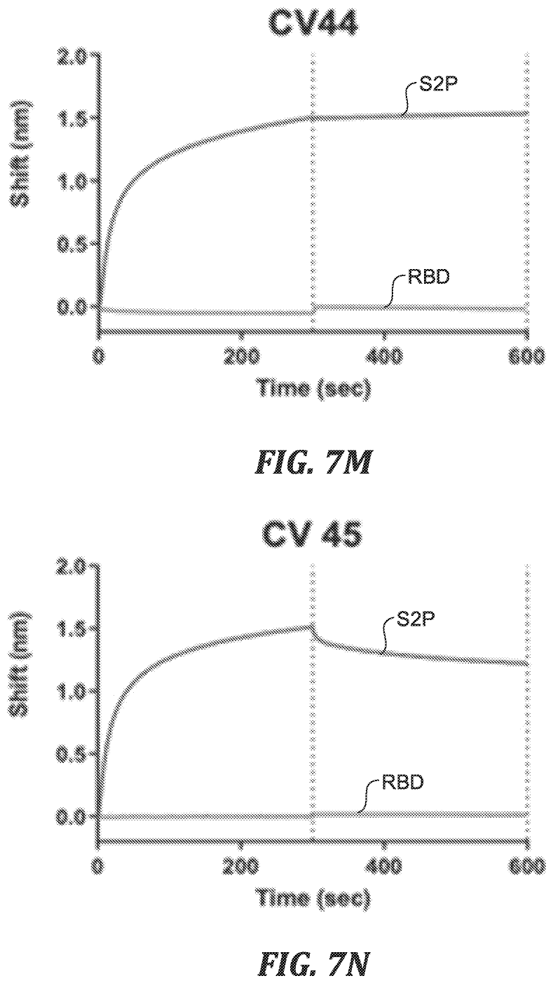

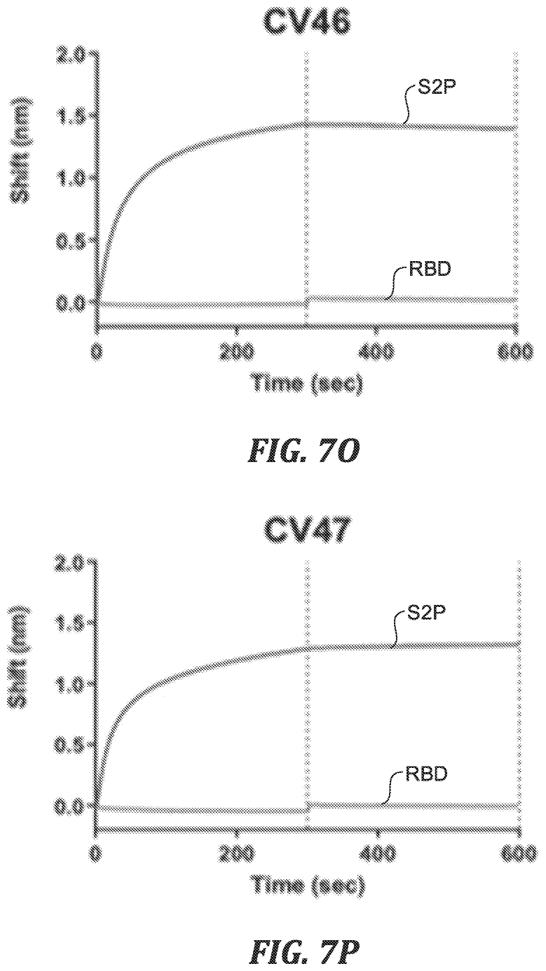

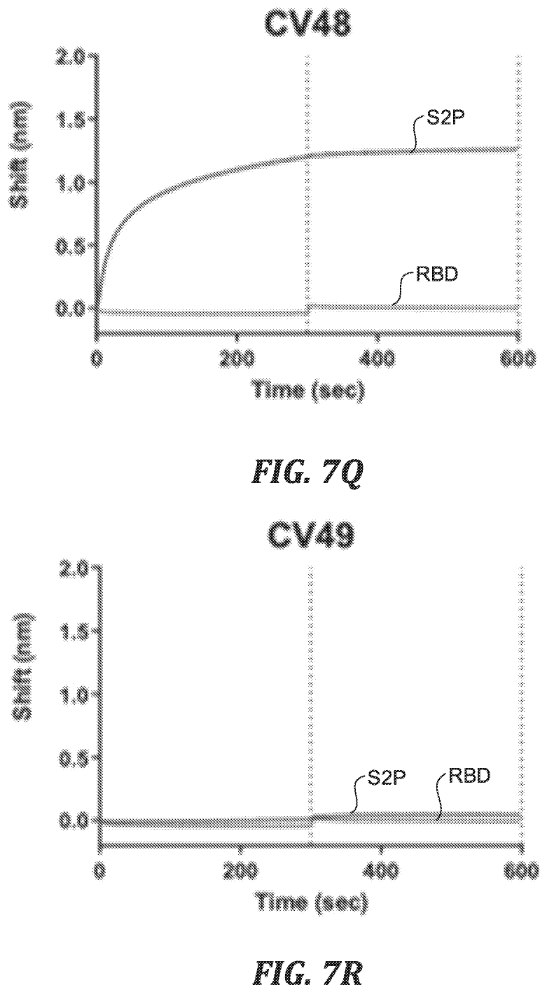

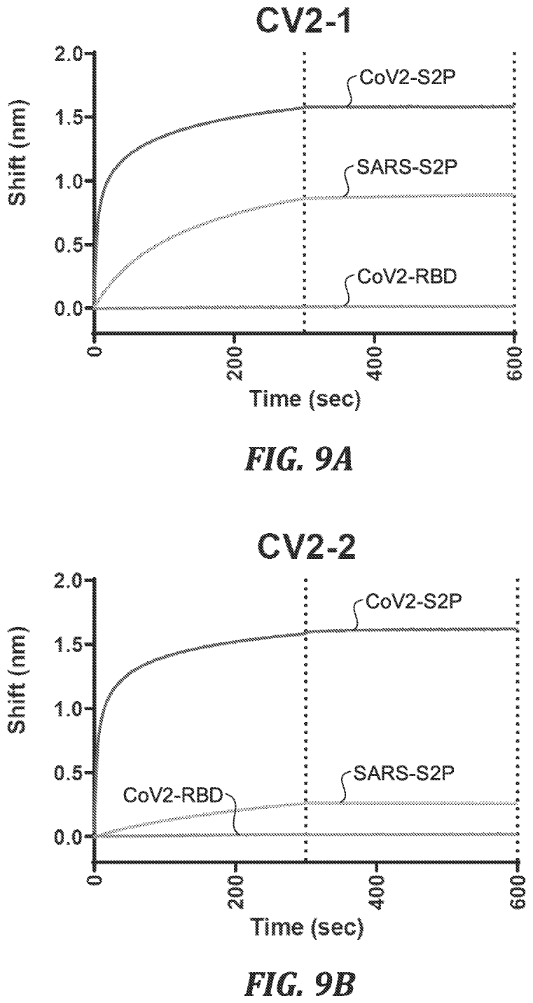

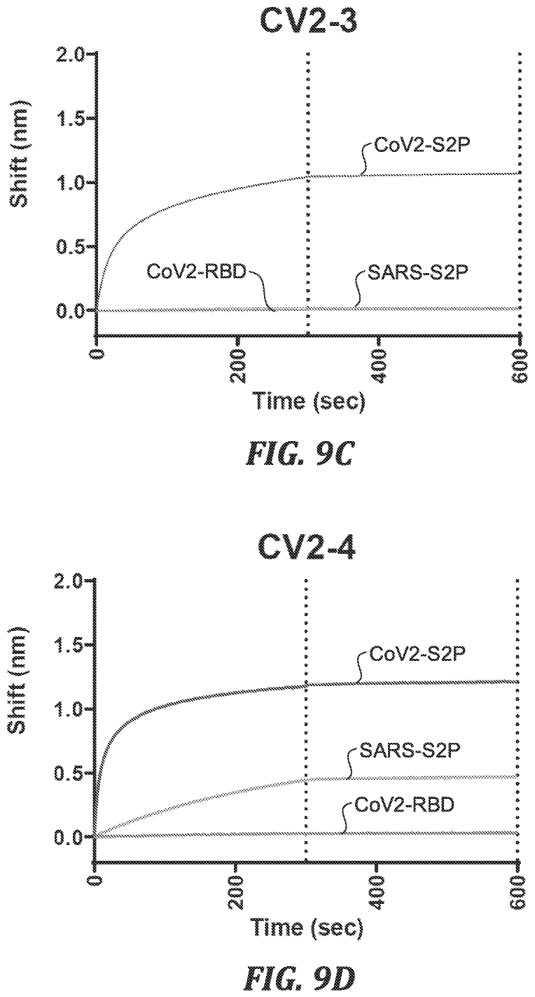

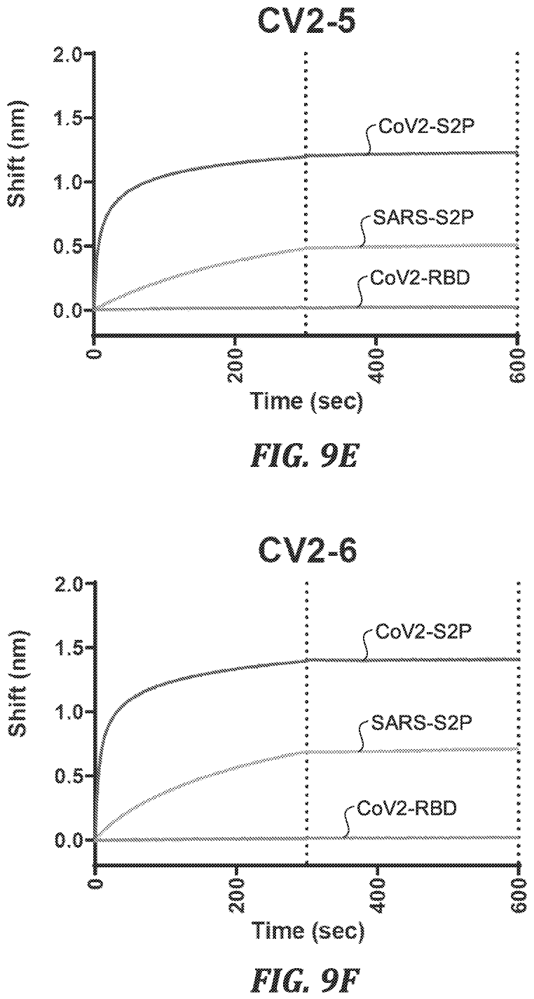

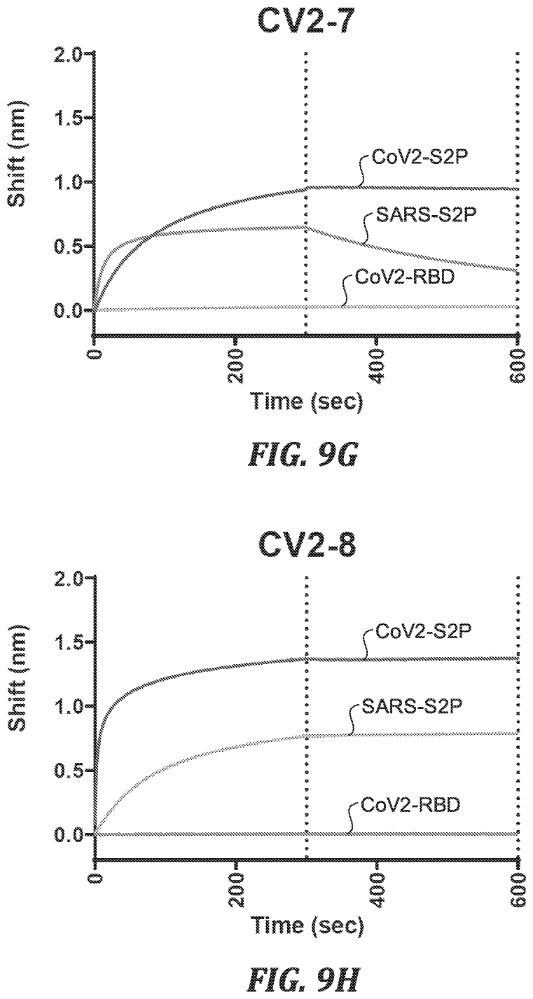

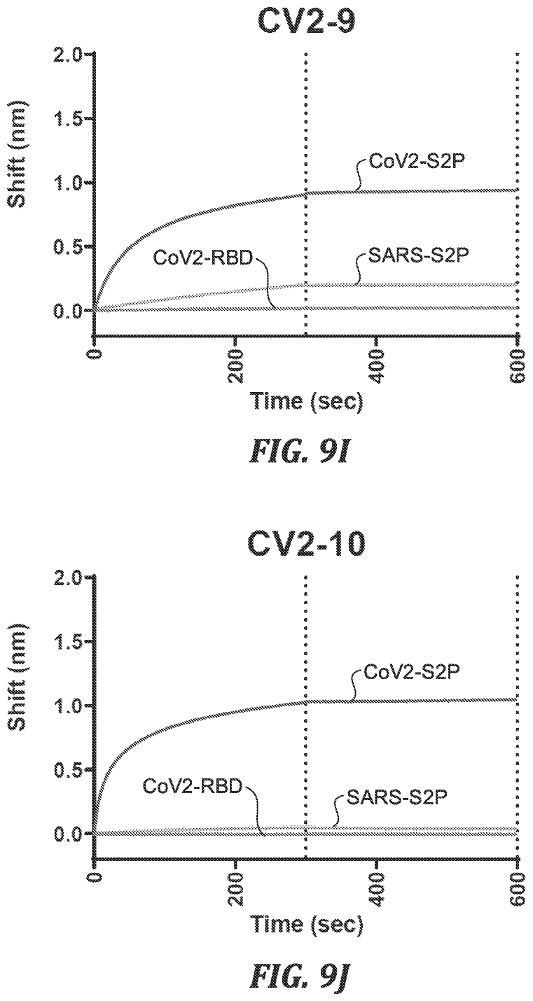

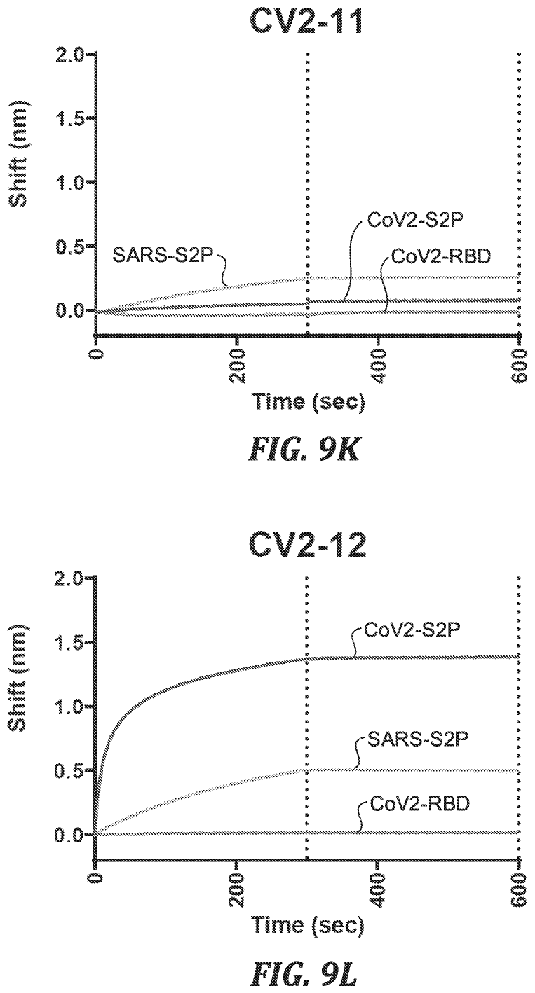

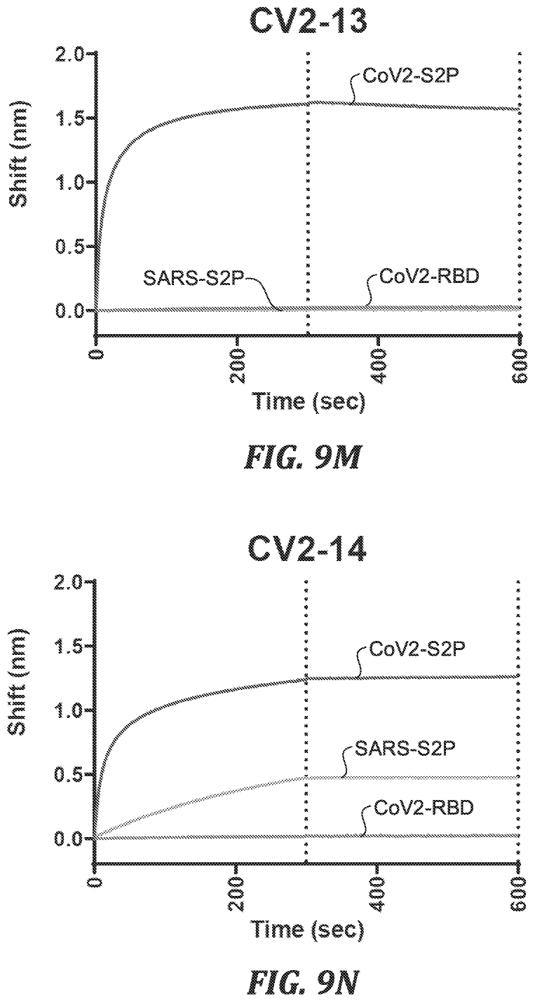

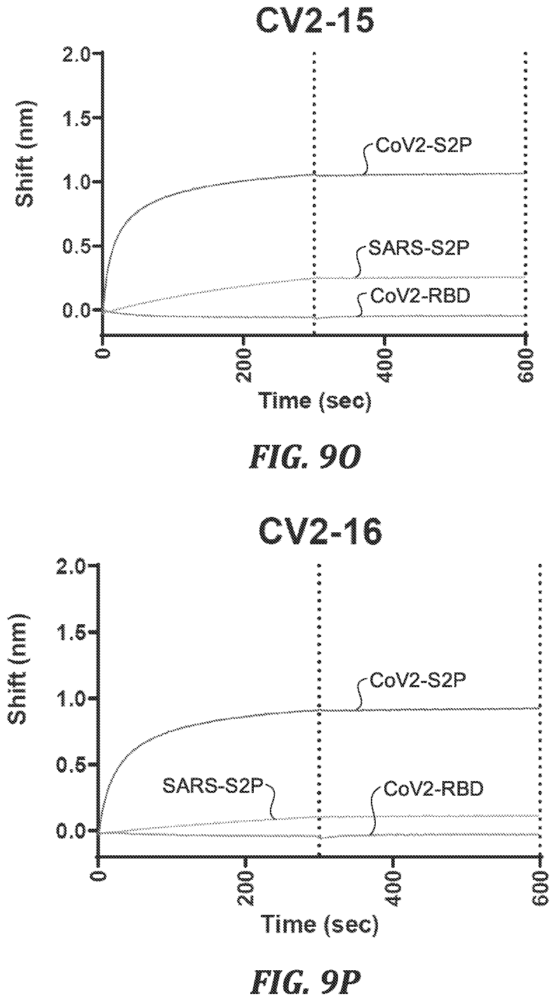

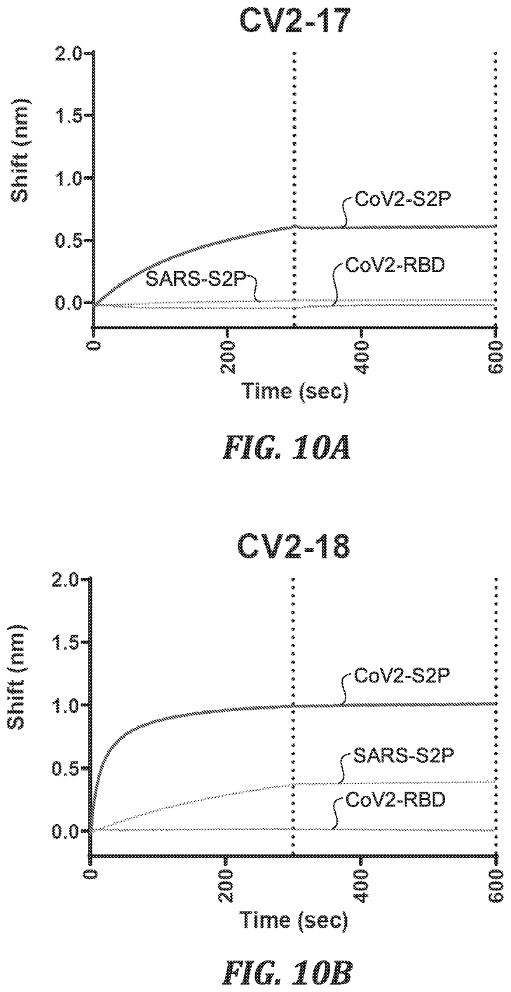

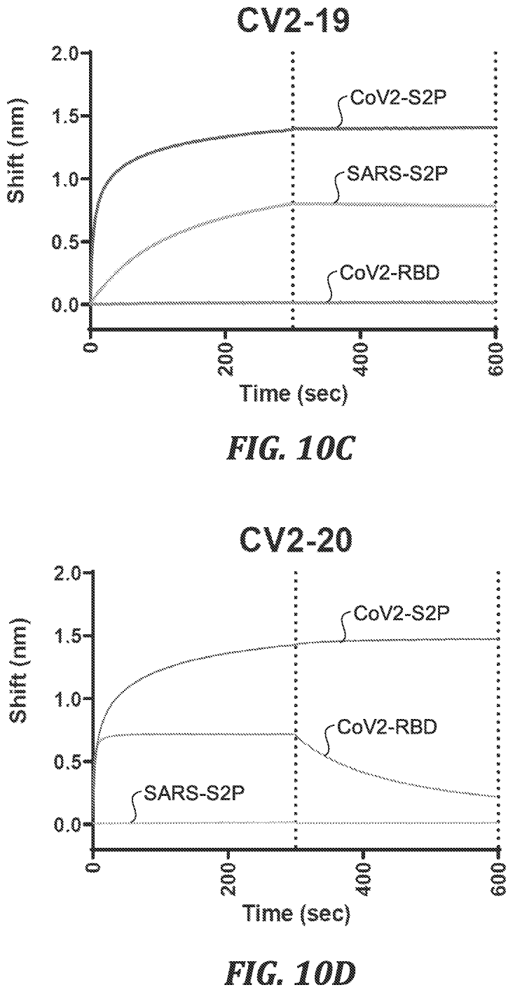

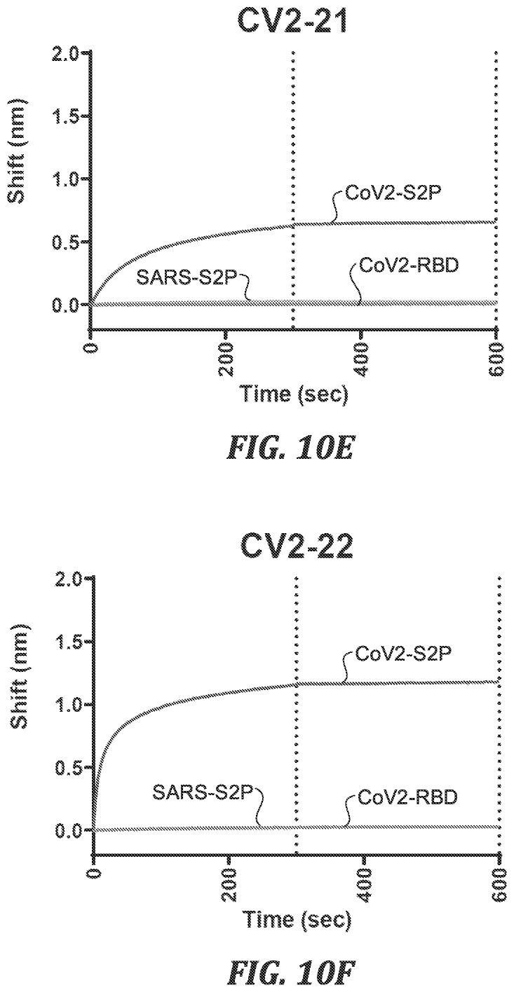

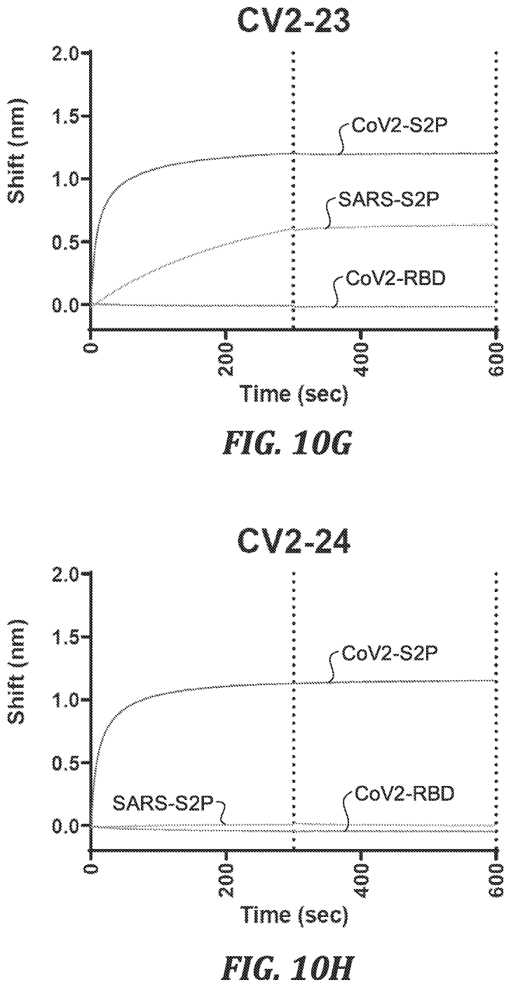

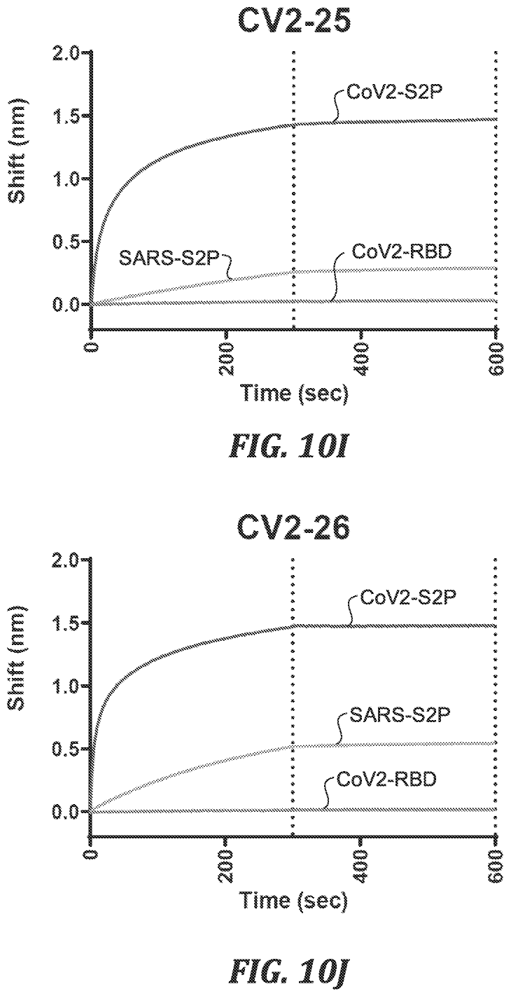

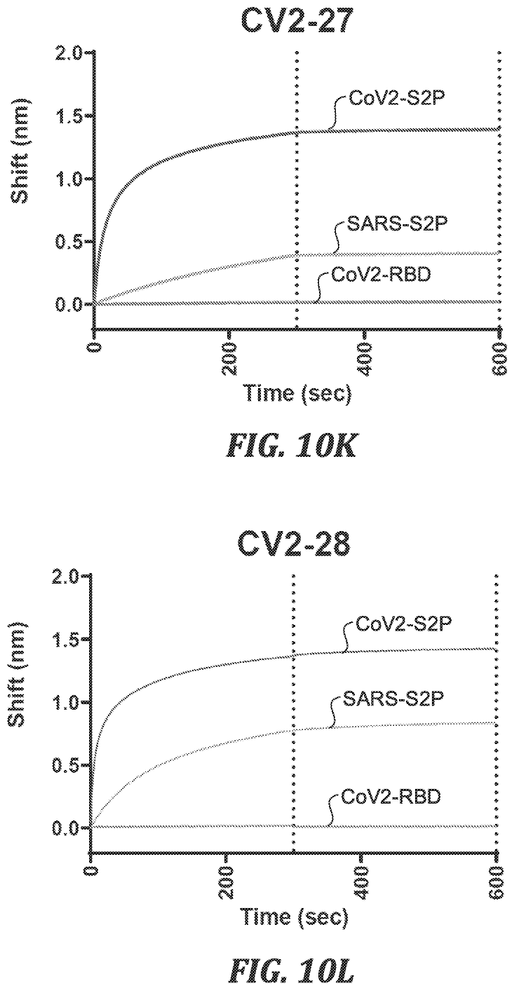

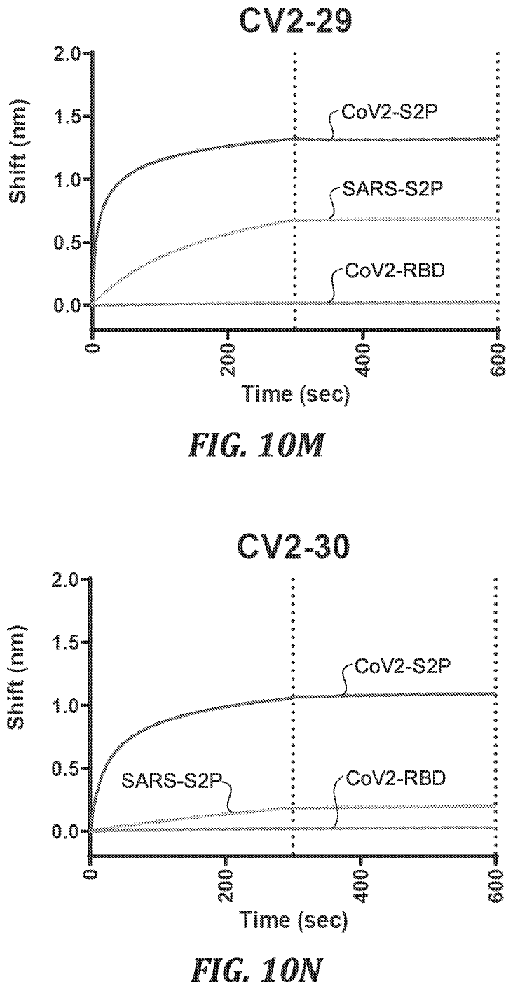

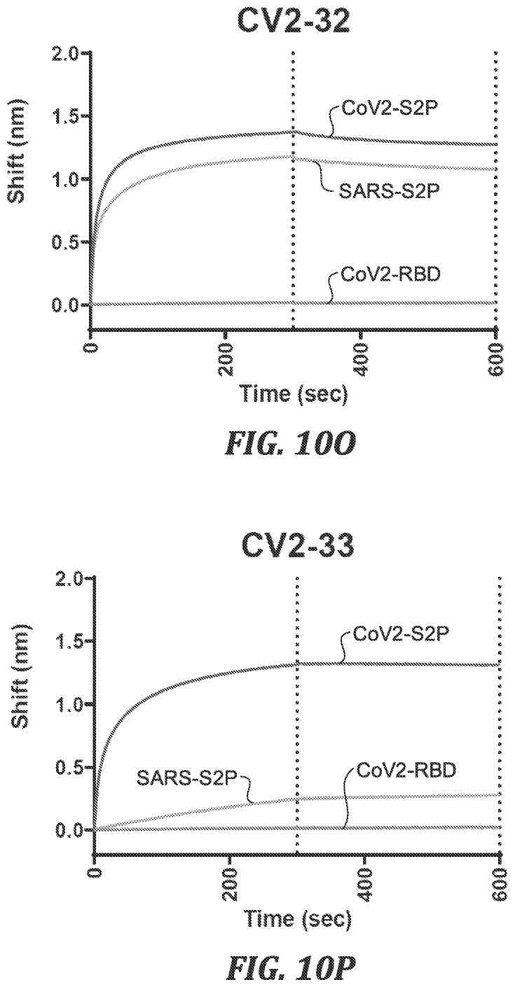

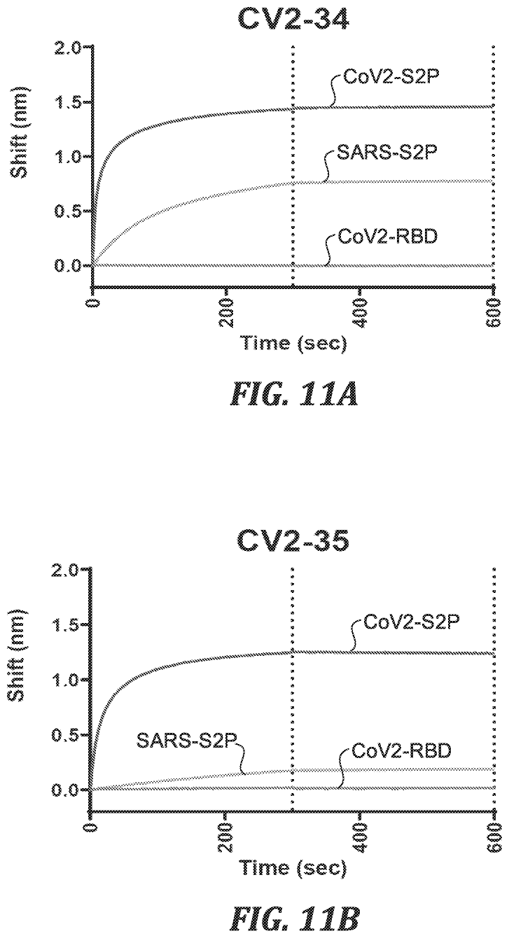

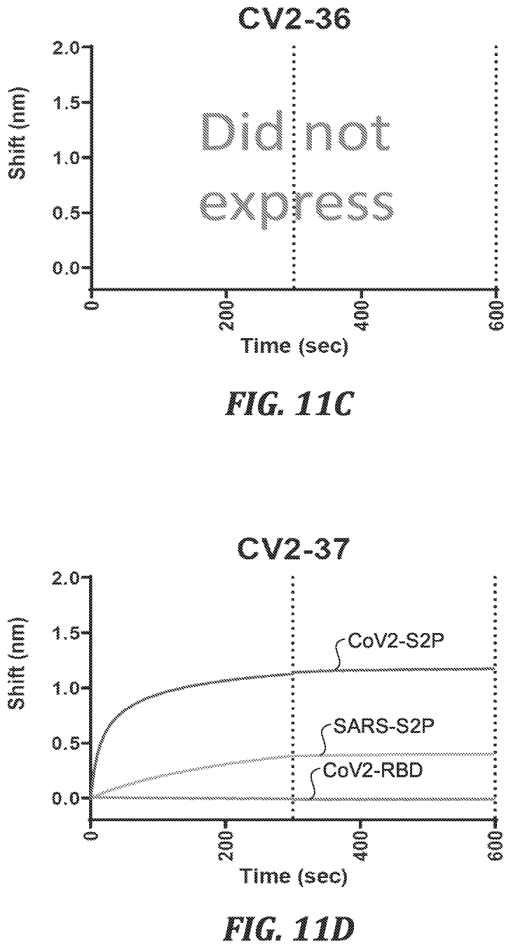

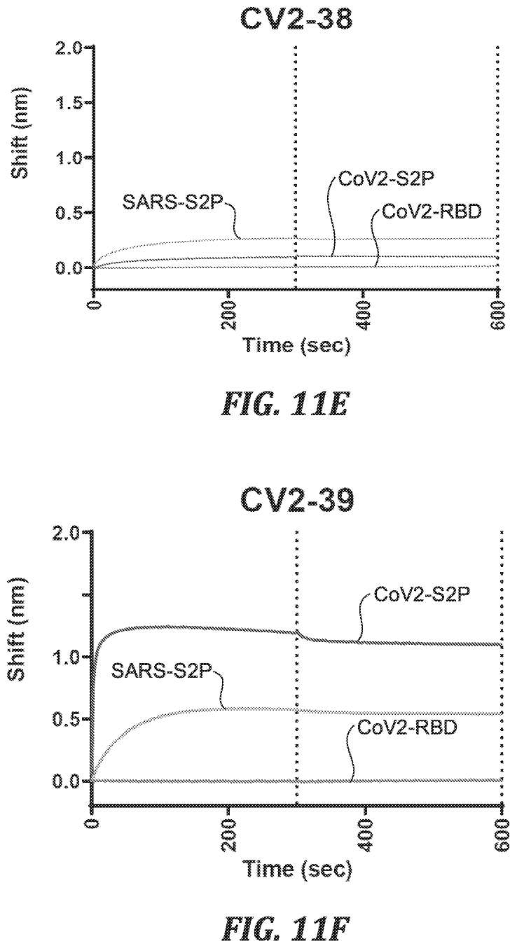

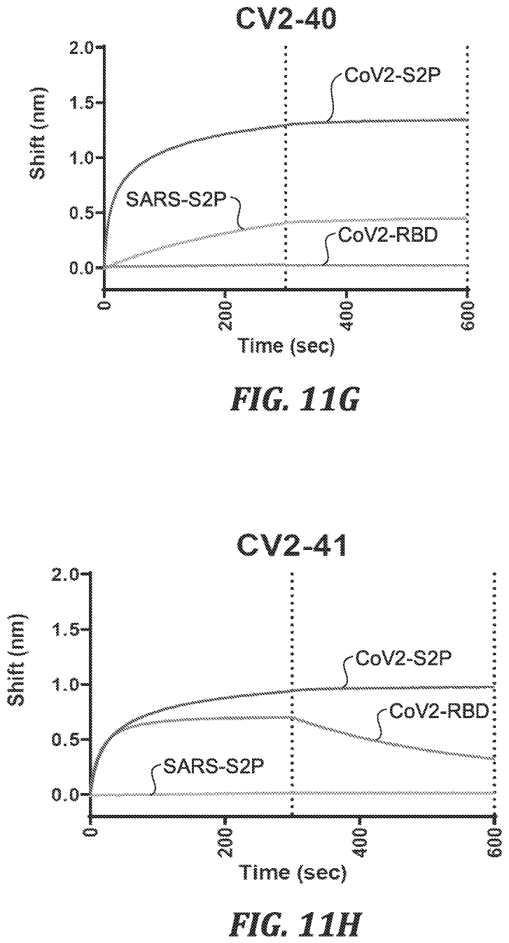

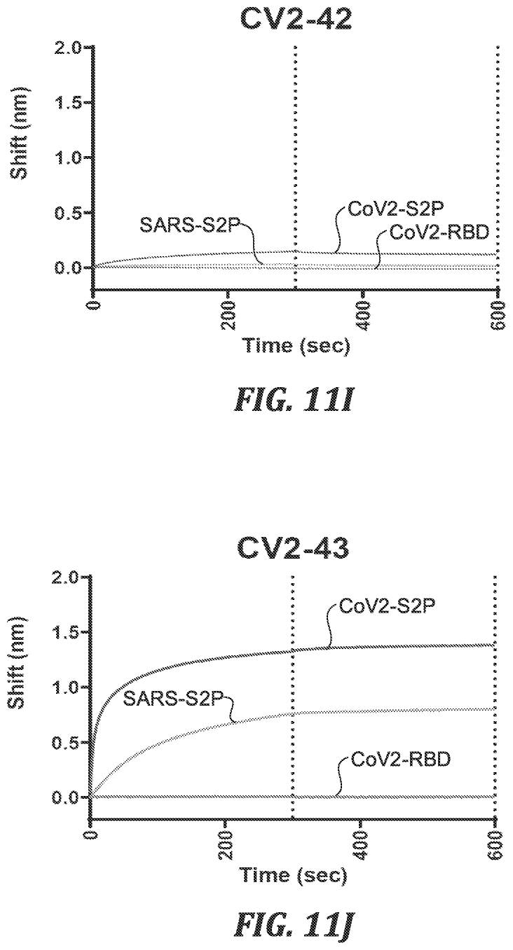

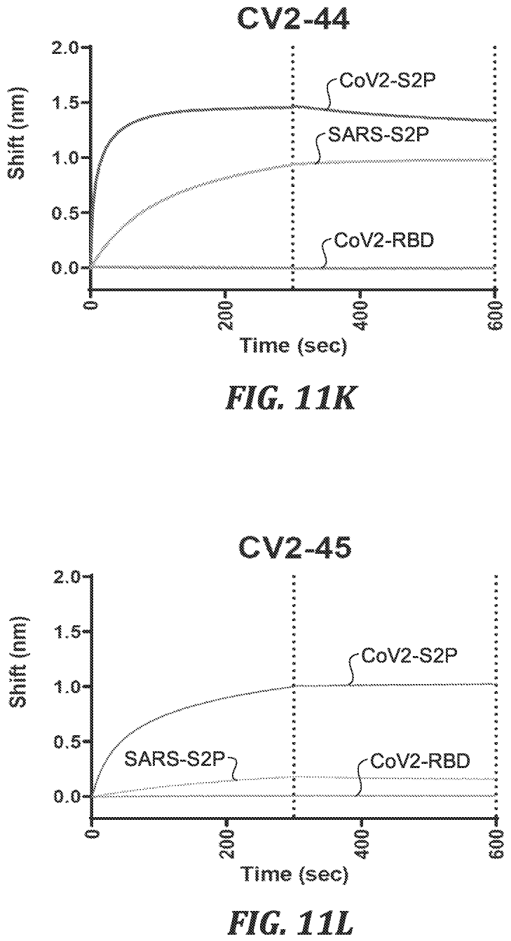

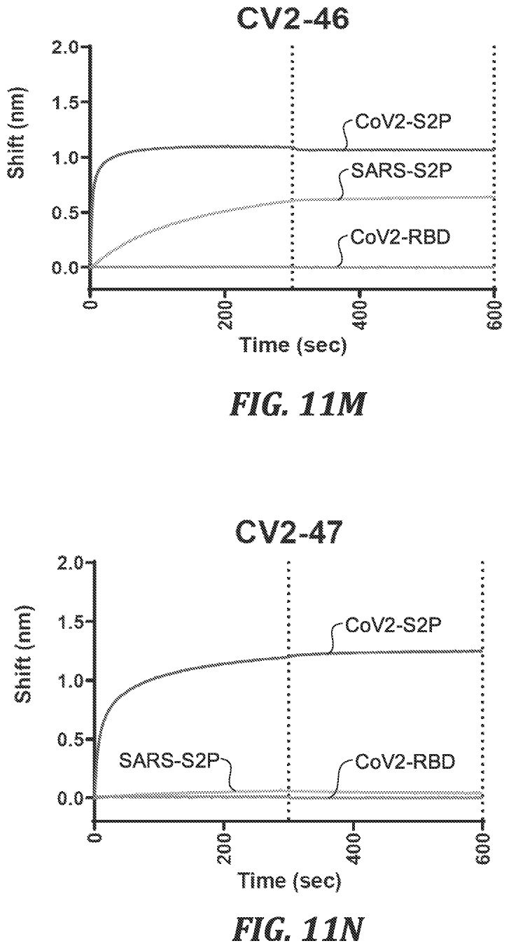

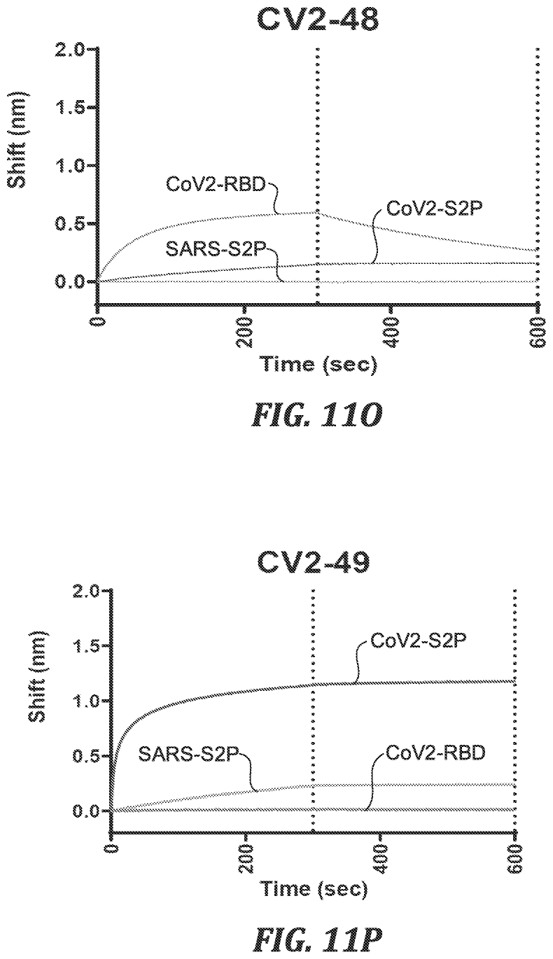

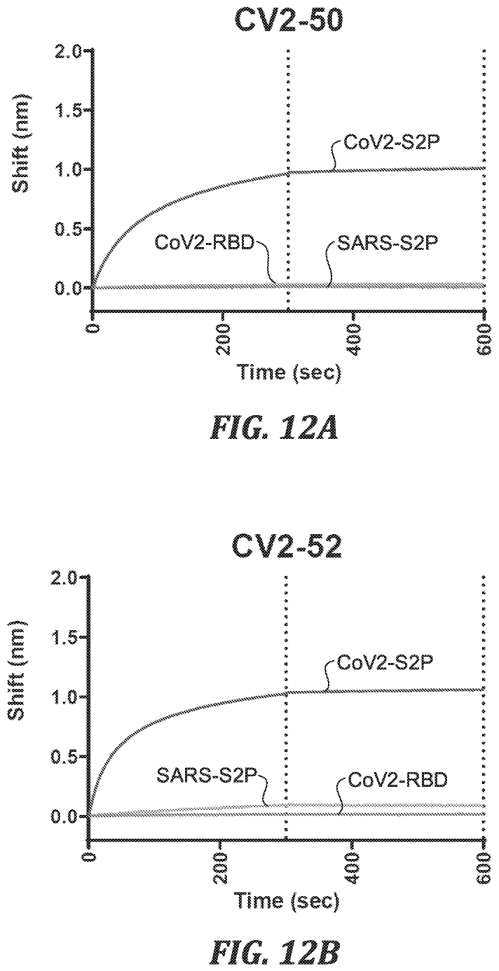

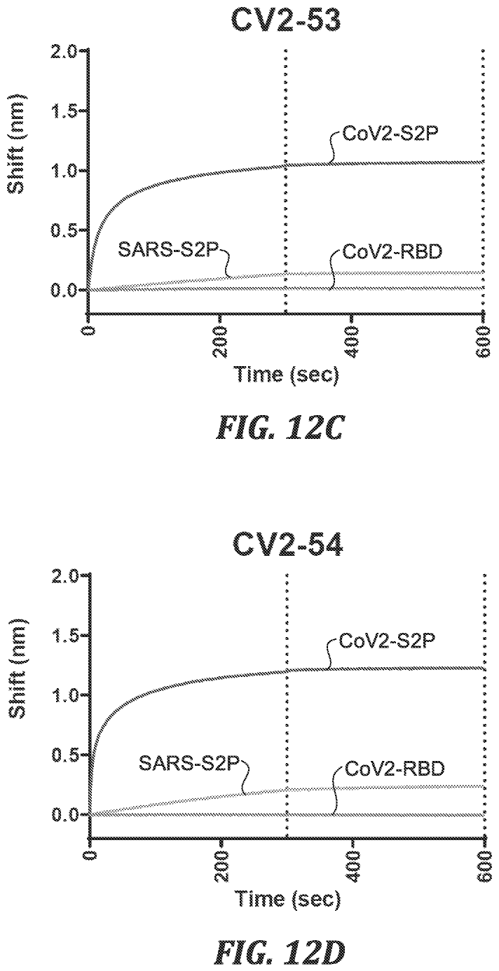

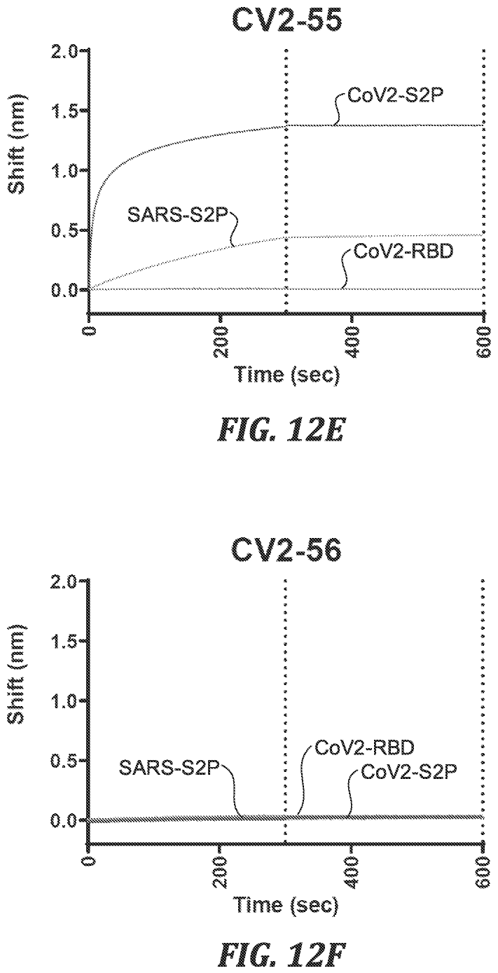

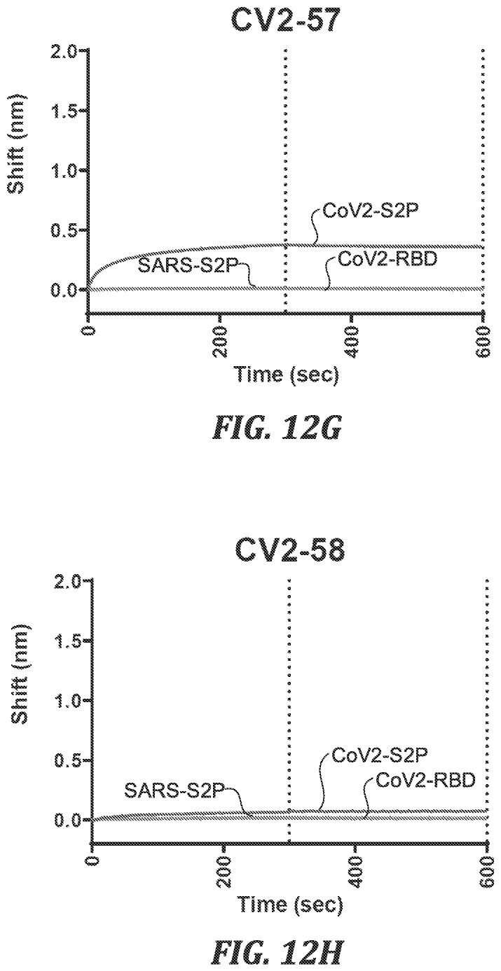

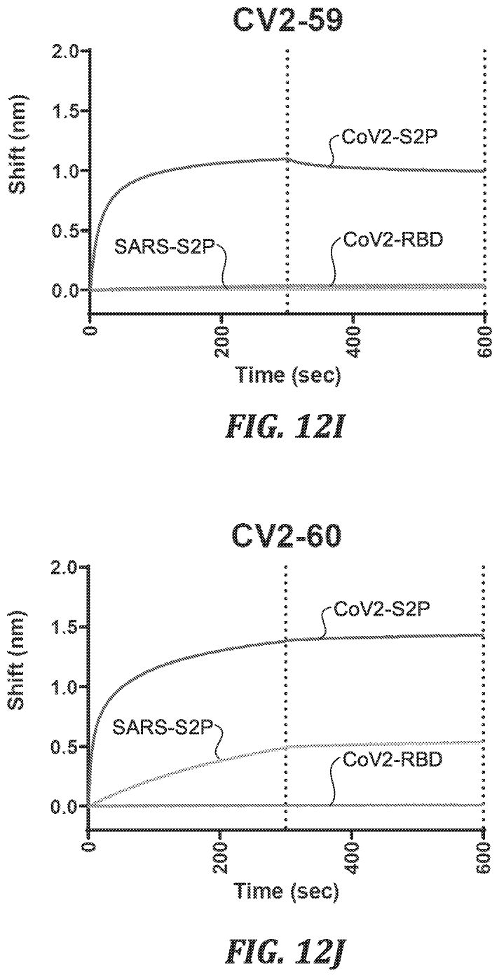

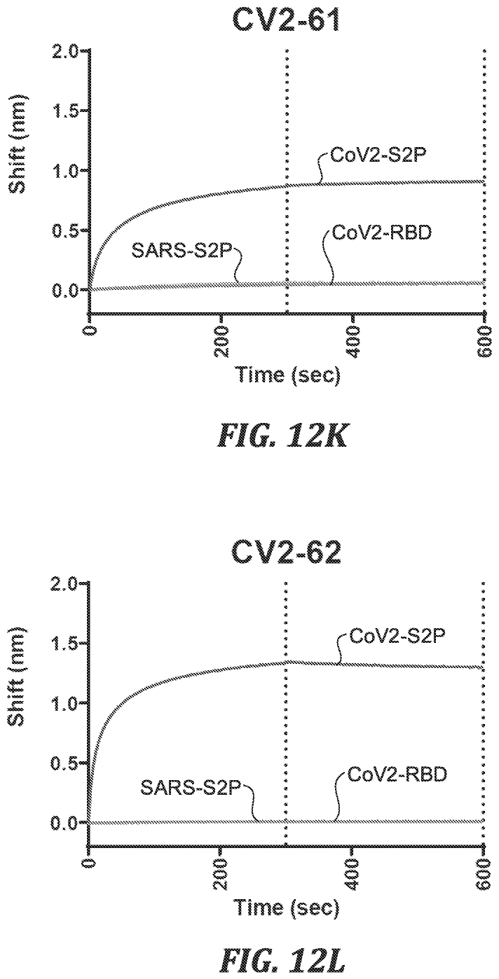

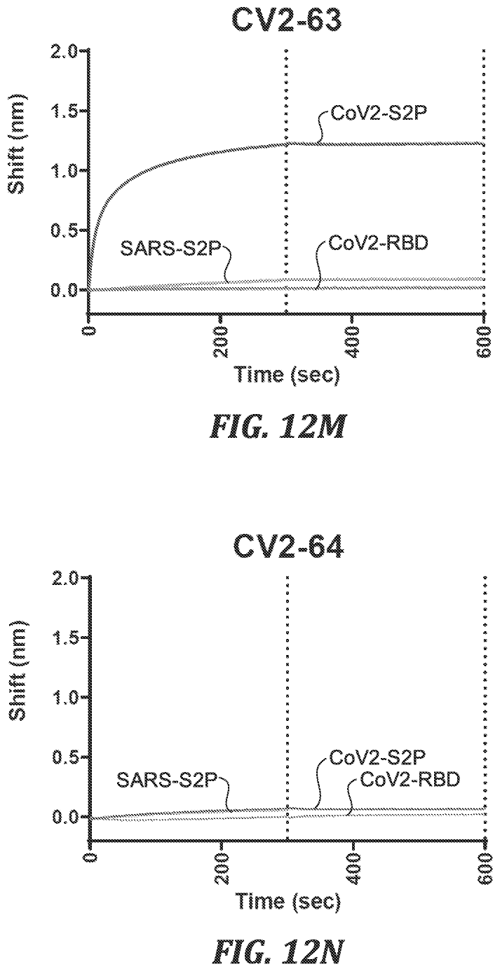

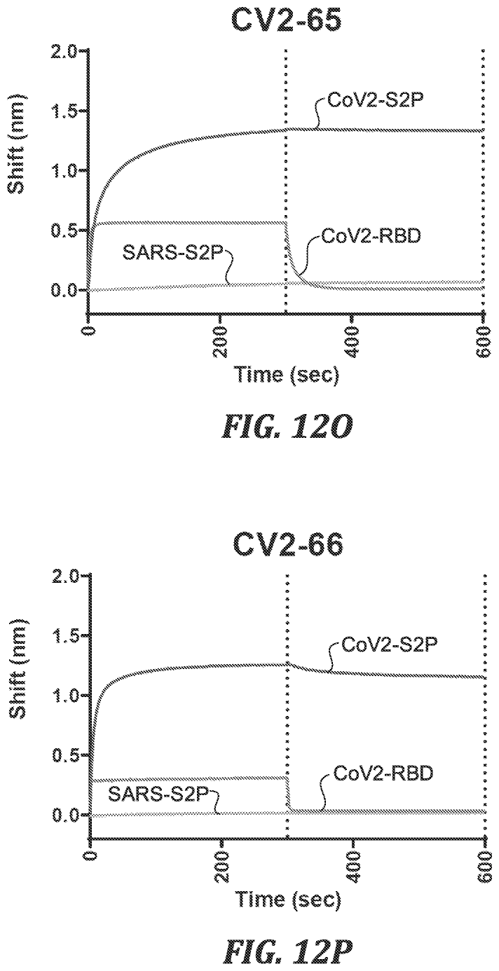

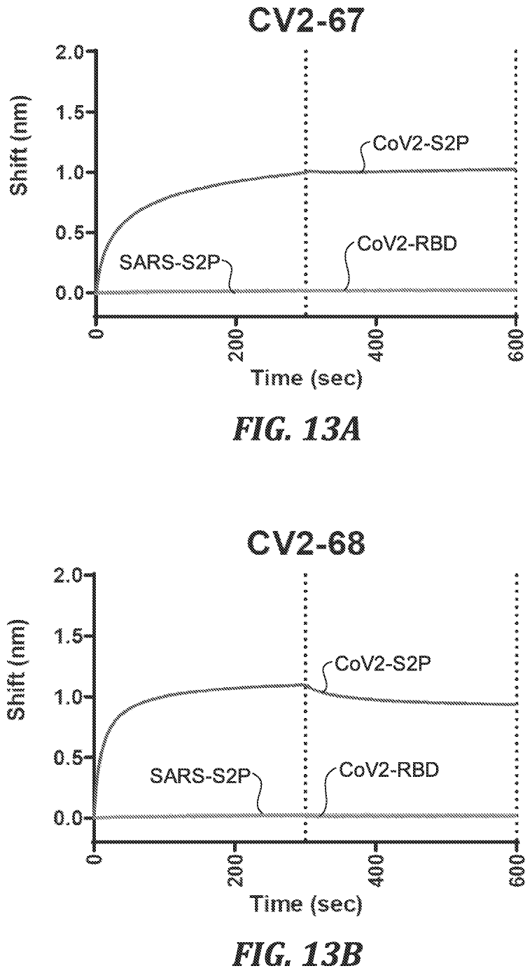

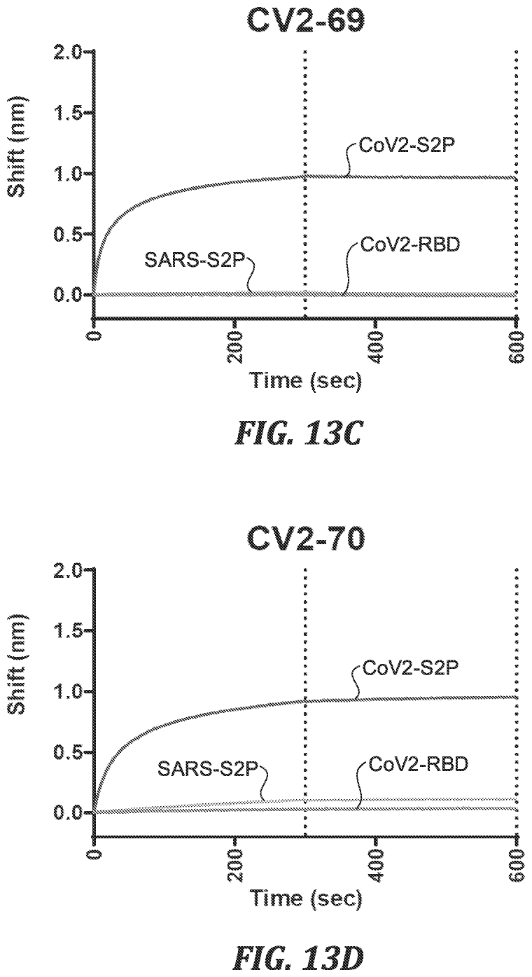

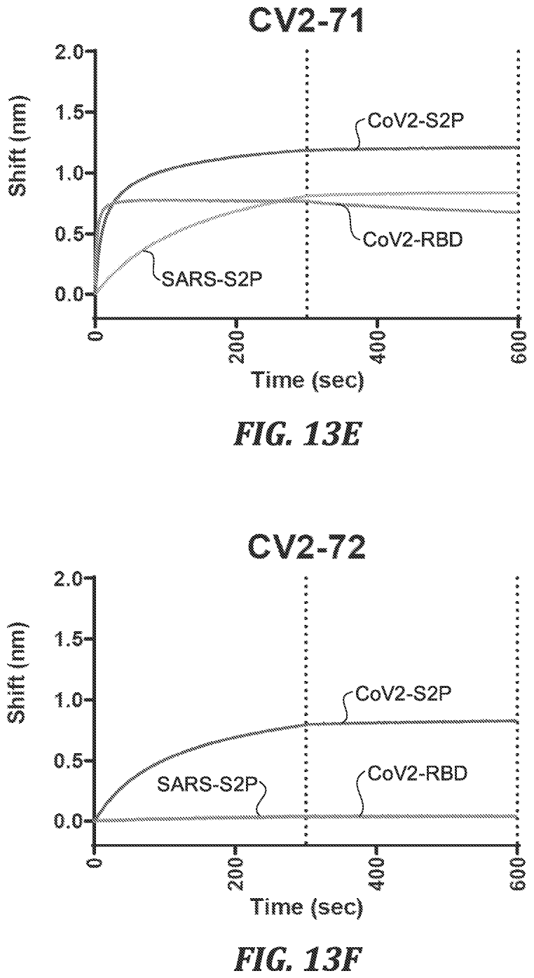

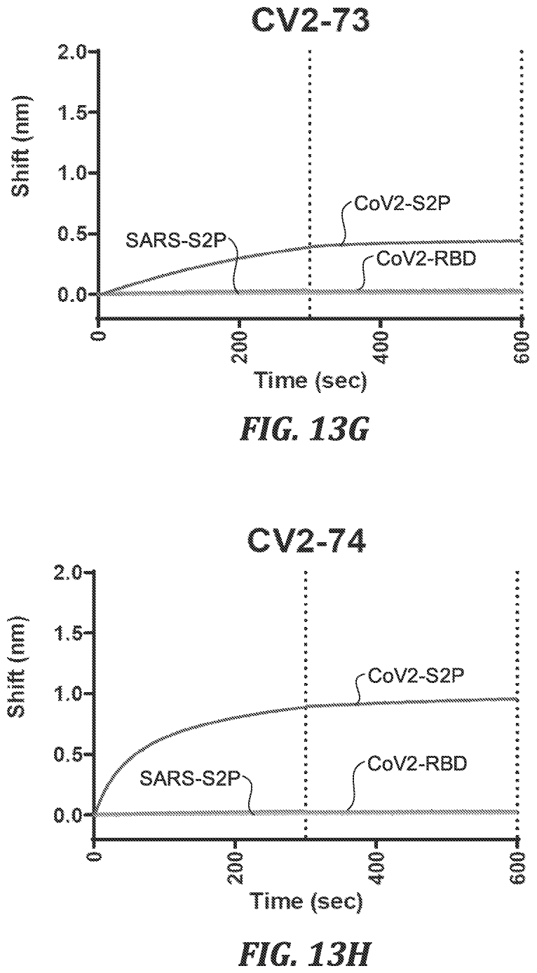

The binding of several mAbs initially isolated from the initial COVID-19 infected patient (45 mAbs referred to as: CV1-CV25, CV27, CV30-CV34, and CV36-CV49) were evaluated for binding to S2P and RBD using the strategy illustrated in FIGS. 4A and 4B. COVID-19-derived mAbs were loaded onto an anti-human Fc probe and dipped in the SARS-CoV2 recombinant envelope proteins to measure binding using biolayer interferometry (BLI): CoV2-RBD (receptor binding domain) and CoV2-S2P (extracellular portion of the trimeric envelope spike). Of the mAbs tested, most bound to S2P. mAbs CV5, CV30, and CV43 bound both S2P and RBD. The RBD and S2P binding results are shown in FIGS. 5A-7R. Additionally, several mAbs (identified as CV2-1-CV2-30, CV2-32-CV2-50, CV2-52-CV2-75) were similarly isolated from additional COVID-19 patients and were similarly tested for binding to RBD and S2P, as described above, and for SARS-S2P protein. mAbs CV2-7, CV2-20, CV2-41, CV2-65, CV2-66, CV2-71, and CV2-75 bound both S2P and RBD. The RBD, S2P, and SARS-S2P binding results are shown in shown in FIGS. 9A-13I.

Neutralizing Activity of Exemplary Anti-CoV2 Monoclonal Antibodies

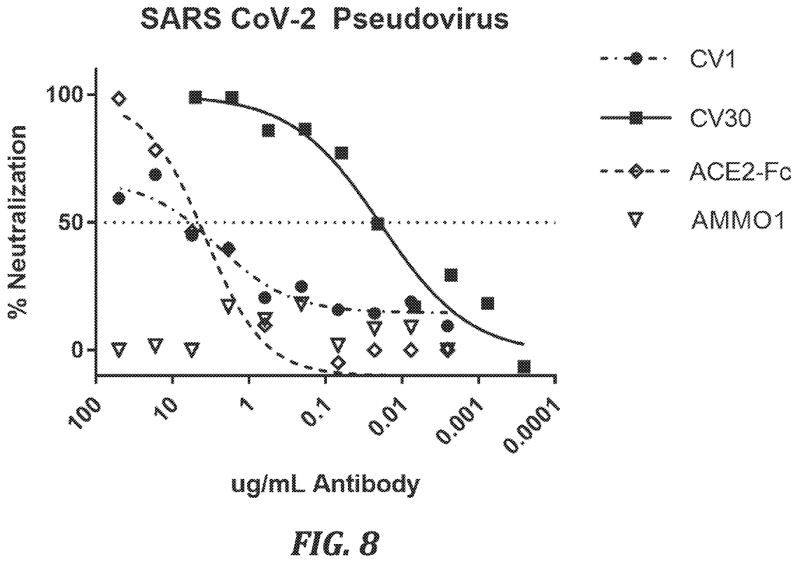

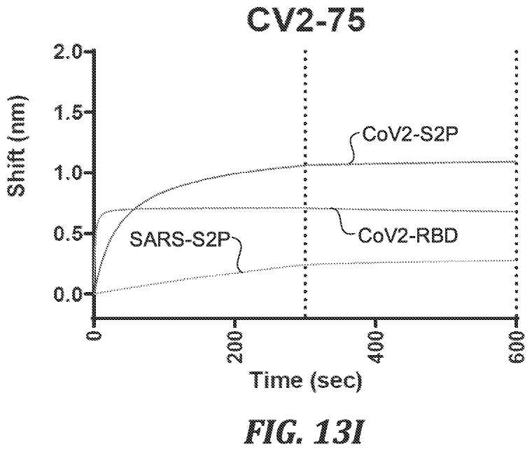

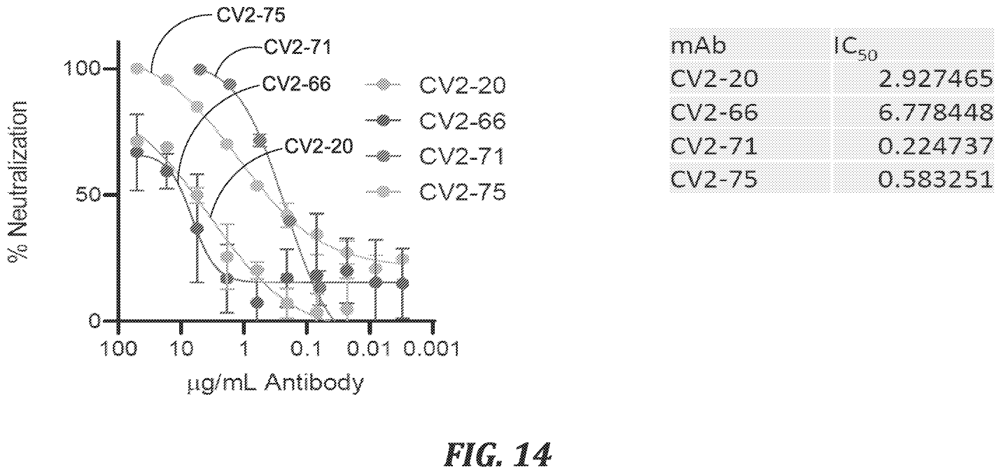

HIV-1 derived lentiviral particles pseudotyped with the SARS CoV-2 Spike (S) protein capable of delivering a luciferase reporter gene were mixed with the indicated monoclonal antibodies, incubated for 1 hour and then added to cells stably expressing ACE2; the SARS CoV-2 receptor. The ACE2 ectodomain fused to an IgG1 Fc (ACE2-Fc), which acts as a competitive inhibitor of spike-binding to cell-surface ACE2, and the anti-EBV gH/gL antibody AMMO1 were included as positive and negative controls, respectively. 48 hours later the cells were lysed, and luciferase activity was measured. Neutralizing activity is reported as the reduction in infectivity in the presence of the mAb relative to the infectivity in the absence of mAb. The viral particles and the cell line were generated as described in Crawford, K. H. D., et al. Protocol and reagents for pseudotyping lentiviral particles with SARS-CoV-2 Spike protein for neutralization assays. Viruses 2020, 12(5), 513, incorporated herein by reference in its entirety. The exemplary results are shown in FIGS. 8 and 14.

While illustrative embodiments have been illustrated and described, it will be appreciated that various changes can be made therein without departing from the spirit and scope of the invention.

1. An antibody or derivative thereof that binds to an epitope in the viral envelope spike protein (S2P) of a Severe Acute Respiratory Syndrome Coronavirus 2 (SARS-CoV-2), wherein the antibody or derivative thereof comprises a heavy chain variable domain (V_{H}) comprising an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 and a light chain variable domain (V_{L}) comprising an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242.

2. The antibody or derivative thereof of claim 1, wherein the antibody is a monoclonal antibody or a derivative thereof.

3. The antibody or derivative thereof of claim 1, wherein the antibody or a derivative thereof is isolated.

4. The antibody or derivative thereof of claim 1, wherein the amino acid sequence of the heavy chain variable domain (V_{H}) is represented by SEQ ID NO: N and the amino acid sequence of the light chain variable domain (V_{L}) is represented by SEQ ID NO: N+1.

5. The antibody or derivative thereof of claim 1, wherein the antibody or derivative thereof binds to an epitope of the receptor binding domain (RBD) of a Severe Acute Respiratory Syndrome Coronavirus 2 (SARS-CoV-2).

6. The antibody or derivative thereof of claim 1, wherein the antibody or derivative thereof neutralizes SARS-CoV-2.

7. The antibody or derivative thereof of claim 1, wherein the antibody or derivative thereof inhibits viral and cell membrane fusion.

8. The antibody or derivative thereof of claim 1, wherein the antibody or derivative thereof is a fully human antibody.

9. The antibody or derivative thereof of claim 1, wherein the antibody or derivative thereof comprises a heavy chain variable domain (VH) comprising amino acid sequence SEQ ID NO: 1 and a light chain variable domain (VL) comprising amino acid sequence SEQ ID NO: 2.

10. The antibody or derivative thereof of claim 1, wherein the antibody or derivative thereof comprises: a heavy chain variable domain (V_{H}) comprising amino acid sequence SEQ ID NO: 9 and a light chain variable domain (V_{L}) comprising amino acid sequence SEQ ID NO: 10; a heavy chain variable domain (V_{H}) comprising amino acid sequence SEQ ID NO: 55 and a light chain variable domain (V_{L}) comprising amino acid sequence SEQ ID NO: 56; a heavy chain variable domain (V_{H}) comprising amino acid sequence SEQ ID NO: 81 and a light chain variable domain (V_{L}) comprising amino acid sequence SEQ ID NO: 82; a heavy chain variable domain (V_{H}) comprising amino acid sequence SEQ ID NO: 109 and a light chain variable domain (V_{L}) comprising amino acid sequence SEQ ID NO: 110; a heavy chain variable domain (V_{H}) comprising amino acid sequence SEQ ID NO: 135 and a light chain variable domain (V_{L}) comprising amino acid sequence SEQ ID NO: 136; a heavy chain variable domain (V_{H}) comprising amino acid sequence SEQ ID NO: 175 and a light chain variable domain (V_{L}) comprising amino acid sequence SEQ ID NO: 176; a heavy chain variable domain (V_{H}) comprising amino acid sequence SEQ ID NO: 221 and a light chain variable domain (V_{L}) comprising amino acid sequence SEQ ID NO: 222; a heavy chain variable domain (V_{H}) comprising amino acid sequence SEQ ID NO: 223 and a light chain variable domain (V_{L}) comprising amino acid sequence SEQ ID NO: 224; a heavy chain variable domain (V_{H}) comprising amino acid sequence SEQ ID NO: 233 and a light chain variable domain (V_{L}) comprising amino acid sequence SEQ ID NO: 234; or a heavy chain variable domain (V_{H}) comprising amino acid sequence SEQ ID NO: 241 and a light chain variable domain (V_{L}) comprising amino acid sequence SEQ ID NO: 242.

11. A composition comprising the antibody or derivative thereof of claim 1 and optionally a carrier.

12. A method of preventing or treating a disease or disorder caused by a severe acute respiratory syndrome coronavirus 2 (SARS-CoV-2), comprising administering to a subject suffering from, or at risk of suffering from, the disease or disorder a therapeutically effective amount of the antibody or derivative thereof of claim 1.

13. The method of claim 12, wherein the method further comprises administering an anti-viral drug, a viral entry inhibitor, or a viral attachment inhibitor.

14. The method of claim 12, wherein the antibody or derivative thereof is administered prior to or after exposure to SARS-CoV-2.

15. A method of detecting the presence of a severe acute respiratory syndrome coronavirus 2 (SARS-CoV-2) in a sample, the method comprising contacting the sample with an antibody or derivative thereof of claim 1 and detecting the presence or absence of an antibody-antigen complex, thereby detecting the presence of a SARS-CoV-2.

16. The method of claim 15, wherein sample is obtained from blood, cheek scraping or swab, nasal swab, saliva, biopsy, urine, feces, sputum, nasal aspiration, or semen.

17. (canceled)

18. A method of delaying the onset of one or more symptoms of severe acute respiratory syndrome coronavirus 2 (SARS-CoV-2) comprising administering to a subject at risk a therapeutically effective amount of the antibody or derivative thereof of claim 1.

19. A nucleic acid comprising a sequence encoding an amino acid sequence of any one of SEQ ID NOS 1-26, 29-38, 41-92, and 95-242.

20. An antigen-binding composition comprising an antibody or antigen-binding fragment thereof that specifically binds to an epitope in the viral envelope spike protein (S2P) of a Severe Acute Respiratory Syndrome Coronavirus 2 (SARS-CoV-2), wherein the antibody or antigen-binding fragment or derivative thereof comprises a heavy chain variable domain (V_{H}) comprising an amino acid sequence selected from the group consisting of SEQ ID NOS: 1, 3, 5, 7, 9, 11, 13, 15, 17, 19, 21, 23, 25, 29, 31, 33, 35, 37, 41, 43, 45, 47, 49, 51, 53, 55, 57, 59, 61, 63, 65, 67, 69, 71, 73, 75, 77, 79, 81, 83, 85, 87, 89, 91, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, and 241 and a light chain variable domain (V_{L}) comprising an amino acid sequence selected from the group consisting of SEQ ID NOS: 2, 4, 6, 8, 10, 12, 14, 16, 18, 20, 22, 24, 26, 30, 32, 34, 36, 38, 42, 44, 46, 48, 50, 52, 54, 56, 58, 60, 62, 64, 66, 68, 70, 72, 74, 76, 78, 80, 82, 84, 86, 88, 90, 92, 96, 98, 100, 102, 104, 106, 108, 110, 112, 114, 116, 118, 120, 122, 124, 126, 128, 130, 132, 134, 136, 138, 140, 142, 1444, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 237, 238, 240, and 242.

21-22. (canceled)

23. The composition of claim 20, wherein the composition comprises two or more of the antibodies or antigen-binding antibody fragments.

24-25. (canceled)
